(19) United States
(12) Patent Application Publication
(10) Pub. No.: US 2025/0313785 A1
(43) Pub. Date: Oct. 9, 2025

(54) Mini-Bioreactor System For 3-Dimensional Organoid Research And Development

(71) Applicant: NEURONEXUS TECHNOLOGIES, INC., Ann Arbor, MI (US)

(72) Inventors: Bencharong Suwarato, Belmont, MA (US); Daryl R. Kipke, Dexter, MI (US); Rio J. Vetter, Van Buren TWP, MI (US); Jamille Farraye Hetke, Brooklyn, MI (US); Carlos Rackham, Plano, TX (US)

(21) Appl. No.: 19/171,021

(22) Filed: Apr. 4, 2025

Related U.S. Application Data
(60) Provisional application No. 63/574,786, filed on Apr. 4, 2024.

(51) Int. Cl. C12M 3/00; C12M 1/00; C12M 1/32; C12M 1/34

(52) U.S. Cl. CPC C12M 21/08; C12M 23/12; C12M 29/06; C12M 41/46

(57) ABSTRACT

Methods, systems, and devices for 3-dimensional organoid research and development. In some embodiments, a bioreactor system for organoids is provided. The bioreactor system may include one or more single-unit bioreactors, a fluid handling system, and a processor. The one or more single-unit bioreactors may include a basket shaped to hold a three-dimensional organoid, a microelectrode array disposed within the basket, and one or more sensors. The fluid handling system may include a first holding chamber for a first fluid, and a dispensing tip coupled to the first holding chamber and configured to deliver the first fluid to at least one of the one or more single-unit bioreactors. The processor may be in communication with the microelectrode array and the one or more sensors and may be configured to receive a first plurality of electrical signals from the one or more sensors.

### PRIORITY

This application claims the benefit of the filing date of U.S. Provisional Application 63/574,786, filed Apr. 4, 2024 and titled “Mini-Bioreactor System For 3-Dimensional Organoid Research And Development,” the entirety of which is incorporated herein by reference.

### TECHNICAL FIELD

The present disclosure relates generally to systems, devices, and methods for cultivating, growing, developing, and/or researching organoids.

### BACKGROUND

Organoids are 3-dimensional (3D) in vitro tissue cultures that are intended to mimic an organ or tissue in vivo. Organoids are generally used for research purposes because they strive to replicate the complexity of an organ on a miniaturized scale without needing to use a live organ or animal. For example, organoids can be used in drug development for drug discovery, drug screening, or precision medicine. Organoids can also be used to perform disease modeling. A wide variety of organs can be studies using organoids, including the brain, heart, lung, liver, and kidney.

However, growing and analyzing 3D tissue cultures is difficult. Organoids do not have structural integrity. Thus, if an organoid is grown on a flat surface, such as a petri dish, its round shape will become flattened due to the gravity. Additionally, neuro signal recording and stimulation is also difficult for organoids because positions of the implanted microelectrodes in the organoid change over time. Thus, the experimental data becomes inconsistent and unreliable. For these reasons, improved systems, devices, and methods for cultivating organoids is needed to perform reliable, long-term, and large-scale research using organoids.

### SUMMARY

One or more embodiments of the present disclosure may include a basket configured to support an organoid. The basket may include a bottom, a first side extending upward from the bottom, and a microelectrode array disposed on the bottom.

In some embodiments, the first side may be hemispherical. In some embodiments, the first side may be cylindrical. In some embodiments, the basket may also include a second side opposite the first side. In some embodiments, the basket may also include one or more perforations in at least said bottom or said first side. In some embodiments, the microelectrode array may include one or more electrodes configured to penetrate the organoid. In some embodiments, the microelectrode array may include one or more electrodes configured to contact a surface of the array.

One or more embodiments of the present disclosure may include a bioreactor system for organoids. The bioreactor system may include one or more single-unit bioreactors, a fluid handling system, and a processor. The one or more single-unit bioreactors may include a basket shaped to hold a three-dimensional organoid, a microelectrode array disposed within the basket, and one or more sensors. The fluid handling system may include a first holding chamber for a first fluid, and a dispensing tip coupled to the first holding chamber and configured to deliver the first fluid to at least one of the one or more single-unit bioreactors. The processor may be in communication with the microelectrode array and the one or more sensors and may be configured to receive a first plurality of electrical signals from the one or more sensors.

In some embodiments, the processor may be further configured to receive a second plurality of electrical signals from the microelectrode array. In some embodiments, the processor may be further configured to send a third plurality of electrical signals to the microelectrode array. In some embodiments, the processor may be further in communication with the fluid handling system. In some embodiments, the processor may be further configured to control the fluid handling system to dispense a particular amount of the first fluid into a first single-unit bioreactor of the one or more single-unit bioreactors. In some embodiments, the fluid handling system may further include a second holding chamber for holding a second fluid and the dispensing tip may be further configured to deliver the second fluid to at least one of the one or more single-unit bioreactors.

Additional aspects, features, and advantages of the present disclosure will become apparent from the following detailed description.

### BRIEF DESCRIPTION OF THE DRAWINGS

The accompanying drawings illustrate implementations of the systems, devices, and methods disclosed herein and together with the description, serve to explain the principles of the present disclosure.

FIG. 1 illustrates a mini-bioreactor system, according to one or more embodiments of the present disclosure.

FIG. 2 illustrates a perspective view of a bioreactor chamber, according to one or more embodiments of the present disclosure.

FIG. 3 illustrates a cross-sectional view of the bioreactor chamber in FIG. 2 along the 3-3′ line.

FIG. 4 illustrates a multi-well plate, according to one or more embodiments of the present disclosure.

FIG. 5A illustrates an assembled view of a multi-well plate, according to one or more embodiments of the present disclosure.

FIG. 5B illustrates an exploded view of the multi-well plate shown in FIG. 5A, according to one or more embodiments of the present disclosure.

FIG. 6 illustrates a flow chart of the layers of the single-unit bioreactor, according to one or more embodiments.

FIG. 7 illustrates a fluid handling system and a multi-well plate, according to one or more embodiments of the present disclosure.

FIG. 8 illustrates a fluid handling system including a robotic dispenser and a multi-well plate, according to one or more embodiments of the present disclosure.

FIG. 9A illustrates an assembled view of a single-unit bioreactor, according to one or more embodiments of the present disclosure.

FIG. 9B illustrates a zoomed-in view of the chamber of the single-unit bioreactor of FIG. 9A.

FIG. 9C illustrates a zoomed-in view of the chamber of the single-unit bioreactor of FIG. 9A with the organoid enclosure removed and an organoid in the basket.

FIGS. 10A-10B illustrate another embodiment of the single-unit bioreactor, according to one or more embodiments of the present disclosure.

FIG. 11A illustrates a perspective side view of a single-unit bioreactor, according to one or more embodiments of the present disclosure.

FIG. 11B illustrates a perspective top view of the single-unit bioreactor of FIG. 11A with the organoid enclosure removed, according to one or more embodiments of the present disclosure.

FIG. 12 illustrates another embodiment of a generally hemispherical organoid basket, according to one or more embodiments of the present disclosure.

FIG. 13A illustrates a basket with a smaller-sized organoid disposed therein, according to one or more embodiments of the present disclosure.

FIG. 13B illustrates the basket of FIG. 13A with a larger-sized organoid disposed therein.

FIG. 13C illustrates a zoomed-in view of the microelectrode array (MEA) of the basket of FIG. 13A with the larger-sized organoid disposed therein.

FIG. 14A illustrates a perspective top view of another embodiment of an organoid basket, according to one or more embodiments of the present disclosure.

FIG. 14B illustrates a zoomed-in view of the MEA of the basket in FIG. 14A.

FIG. 15A illustrates another embodiment of an organoid basket, according to one or more embodiments of the present disclosure.

FIG. 15B illustrates a zoomed-in view of the organoid basket in FIG. 15A.

FIG. 16A illustrates a cross-sectional view of an organoid basket, according to one or more embodiments of the present disclosure.

FIG. 16B illustrates the MEA of the basket in FIG. 16A.

FIG. 16C illustrates a top view of the opening/closing mechanism of the organoid basket in FIG. 16A.

FIG. 17 illustrates an organoid with a section removed, according to one or more embodiments of the present disclosure.

FIG. 18 illustrates an MEA inserted into a target region of an organoid, according to one or more embodiments of the present disclosure.

FIG. 19A is a perspective view of an organoid with one or more MEAs disposed on the surface thereof, according to one or more embodiments of the present disclosure.

FIG. 19B is a cross-sectional view of the organoid in FIG. 19A along the A-A′ line.

FIG. 20 illustrates a cross-sectional view of an embodiment of an MEA including a plurality of penetrating electrodes extending into an organoid, according to one or more embodiments of the present disclosure.

FIG. 21 illustrates another embodiment of a MEA including a plurality of penetrating electrodes extending into an organoid, according to one or more embodiments of the present disclosure.

FIG. 22A illustrates an electrode shank with a sharp tip, according to one or more embodiments of the present disclosure.

FIG. 22B illustrates an electrode shank with a rounded tip, according to one or more embodiments of the present disclosure.

FIG. 23 illustrates an embodiment of an electrode shank, according to one or more embodiments of the present disclosure.

FIG. 24 illustrates a method of inserting a thin or ultrathin electrode into an organoid, according to one or more embodiments of the present disclosure.

FIG. 25A illustrates a penetrating electrode system inserted into an organoid, according to one or more embodiments of the present disclosure.

FIG. 25B illustrates a detailed view of the penetrating electrode system in FIG. 25A.

FIG. 26A illustrates a penetrating electrode inserted into an organoid, according to one or more embodiments of the present disclosure.

FIG. 26B illustrates a side view of the penetrating electrode in FIG. 26A.

FIG. 27A-27B illustrate another embodiment for system for delivering fluids and/or removing waste from the necrotic core of an organoid, according to one or more embodiments of the present disclosure.

FIG. 28 illustrates another embodiment for system for delivering fluids and/or removing waste from the necrotic core of an organoid, according to one or more embodiments of the present disclosure.

These figures will be better understood by reference to the following Detailed Description.

### DETAILED DESCRIPTION

For the purpose of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In addition, this disclosure describes some elements or features in detail with respect to one or more implementations or figures, when those same elements or features appear in subsequent figures, without such a high level of detail. It is fully contemplated that the features, components, and/or steps described with respect to one or more implementations or figures may be combined with the features, components, and/or steps described with respect to other implementations or figures of the present disclosure. For simplicity, in some instances the same or similar reference numbers are used throughout the drawings to refer to the same or like parts.

When growing and/or researching organoids, it may be important to estimate the cellular and acellular state of the organoid across its entire volume without needing to label or damage the organoid. An organoid may include a necrotic core with a low number (i.e. percentage) of viable cells surrounded by an outer layer having a high number (i.e. percentage) of viable cells. Generally, the outer layer is the target for analyzing the organoid because it has a relatively higher percentage of viable cells. Although the effects of the necrotic core on organoid research is not well-understood, reducing the size of the necrotic core and thereby increasing the number of viable cells in the organoid may better mirror the physiological environment. Thus, reducing the size of the necrotic core may yield improved, more accurate, experimental results.

The cellular and/or acellular state of the organoid may be estimated based on one or more properties of the organoid. For example, these properties may include one or more of cell diversities (e.g. by cell type) throughout the cell, cell fraction (which may be the percentage of the organoid's volume that is occupied by cells), cell types, spatial organization, or the pressure, state, or size of the inner necrotic core. In some embodiments, the organoid state can be used to estimate the similarity among a group of organoids such as, for example, similarities between different organoids in the mini-bioreactor system. This may be important because, when performing an experiment on multiple organoids, ensuring that each organoid is similar may improve the accuracy of the experiment by reducing variability among the tests.

However, current methods of growing and/or analyzing organoids may not support long-term growth or incubation of organoids and, thus, may not be able to perform accurate, long-term experiments on organoids. Moreover, current methods may use labeling which may damage the organoid and/or make it more difficult or inaccurate to analyze the organoid.

Recognizing and taking into account the importance and utility of a methodology and system that can provide the improvements described above, the present disclosure aims to provide methods and systems for improving the growth, incubation, development, and/or research of organoids.

One or more aspects of the present disclosure provide devices, systems, and/or methods for analyzing the organoid state and other properties of the organoid to improve research using organoids. One or more embodiments described herein may allow for long-term experiments to be performed on organoids without the use of labeling. Electrophysiological data and/or biomarker closed-loop feedback may be measured to monitor the growth and/or incubation of the organoid and minimize the size of the necrotic core.

In some embodiments, the present disclosure describes a bioreactor system for growing, cultivating, and/or developing 3-dimensional (3D) organoids. In some embodiments, the bioreactor system may be miniaturized (a “mini-bioreactor system”). The mini-bioreactor system may allow research to be performed using the organoids being grown, cultivated, and/or developed by the mini-bioreactor system.

The mini-bioreactor system may include three distinct features. First, the mini-bioreactor system creates and maintains in vivo environment, through closed-loop feedback, for organoids to stay healthy so that accurate and repeatable experiments can be performed over a long period of time. Second, this system can learn from experience and predict outcomes from inputs using Artificial Intelligence (AI). Third, the system can be monitored and interact remotely in real-time through the Internet of Things (IoT).

The mini-bioreactor system may include one or more single-unit bioreactors, each of which is configured to hold an organoid and perform experiments on that organoid. The single-unit bioreactor may include an organoid basket that is shaped to support and hold a 3D organoid while allowing nutrients to contact the organoid and waste to be removed from it. A micro-electrode array may be disposed on the basket such that it contacts the organoid. One or more electrodes or microelectrodes on the array may be configured to penetrate the organoid. The micro-electrode array may be capable of measuring one or more properties that can be used to estimate the organoid state.

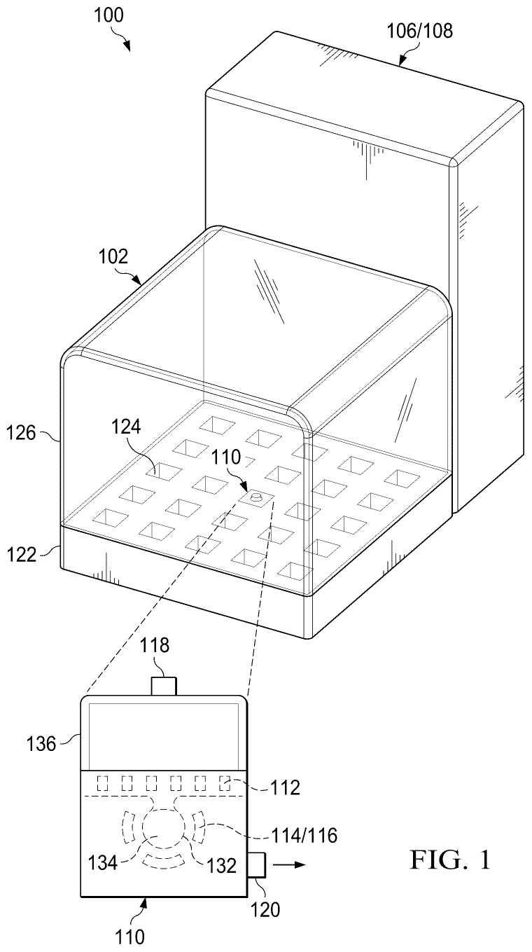

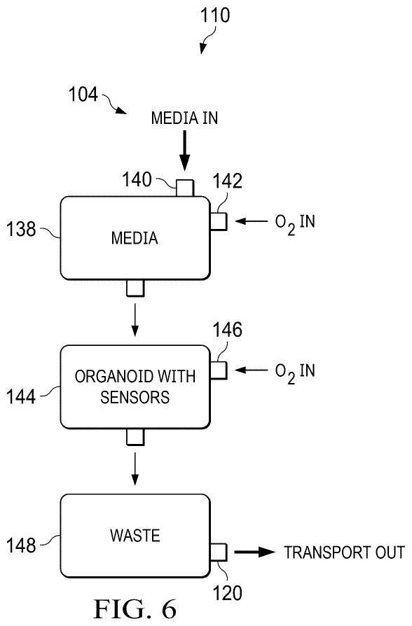

FIG. 1 illustrates a mini-bioreactor system 100, according to one or more embodiments of the present disclosure. The mini-bioreactor system 100 may include four parts or sub-systems that communicate with each other. These sub-systems may include a bioreactor chamber 102, a fluid handling system 104 (shown in FIGS. 6-8), a computer system 106, and a graphical user interface (GUI) 108.

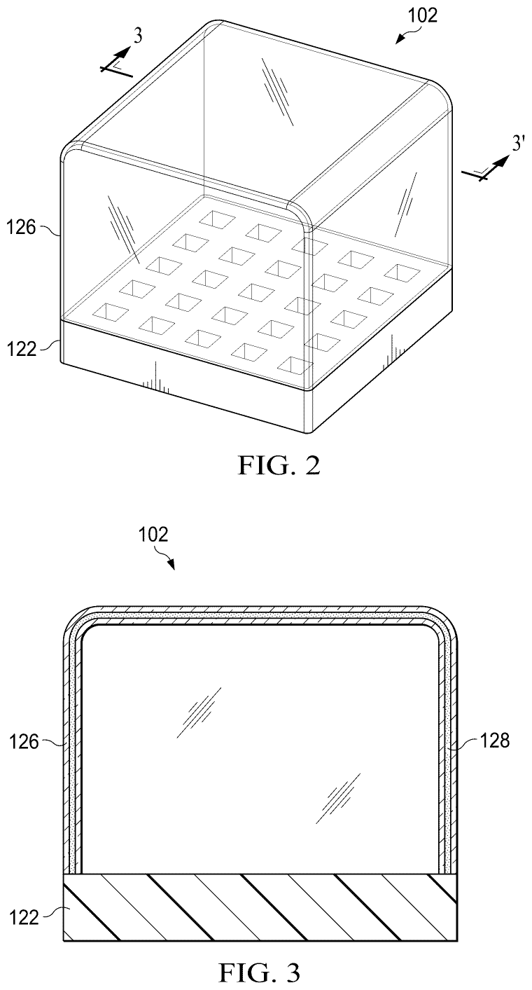

FIG. 2 illustrates a perspective view of a bioreactor chamber 102, according to one or more embodiments of the present disclosure. FIG. 3 illustrates a cross-sectional view of the bioreactor chamber 102 in FIG. 2 along the 3-3 line. The bioreactor chamber 102 may include a multi-well plate 122 that includes one or more individual wells 124 (shown in e.g., FIGS. 1 and 4-5B). The multi-well plate 122 may be covered by a top or chamber enclosure 126. The bioreactor chamber 102 may be insulated. For example, in some embodiments, the chamber enclosure 126 of the bioreactor chamber 102 may include a layer of insulation 128 disposed within it to prevent heat from entering or escaping the bioreactor chamber 102.

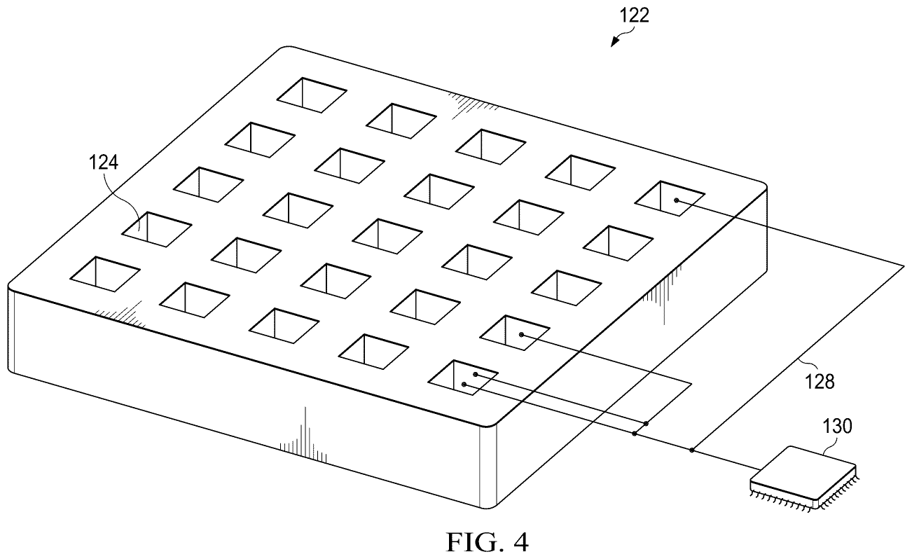

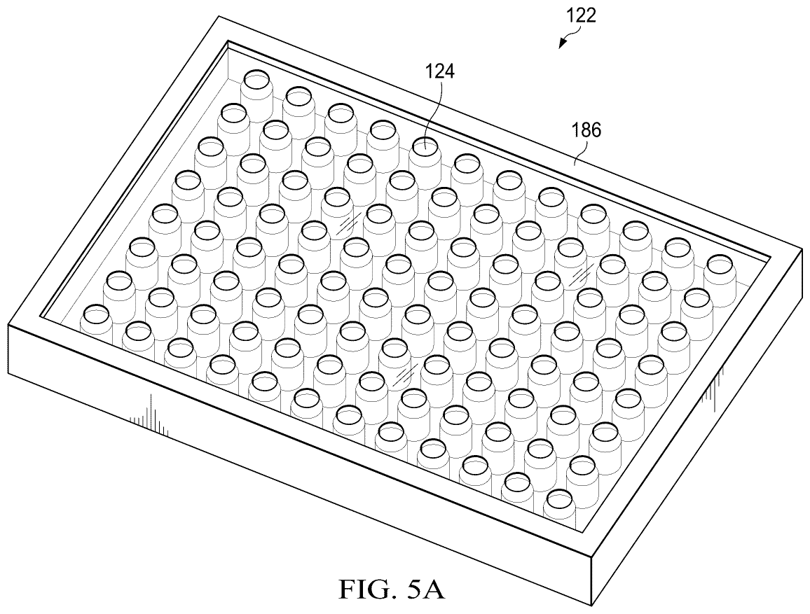

FIG. 4 illustrates a multi-well plate 122, according to one or more embodiments of the present disclosure. Each individual well 124 of the multi-well plate 122 may be connected to an electricity source. For example, each individual well 124 may be connected to an electricity source via electrical lead lines or electrical wires 128. The individual wells 124 may be connected to each other in series and/or in parallel. The individual wells 124 may be connected to a processor 130 that sends electrical signals to and/or receives electrical signals from each individual well 124. The multi-well plate 122 may include any suitable number of individual wells 124. For example, the multi-well plate 122 may have a 5×5 array of individual wells, a 10×10 array of individual wells, a 50×50 array of individual wells, a 100×100 array of individual wells, or an 8×12 array of individual wells.

In some embodiments, the multi-well plate 122 may include multiple processors 130 (or microchips). In some embodiments, each processor 130 may be communicatively coupled to a certain number of individual wells 122. For example, a single processor 130 may be configured to communicate with 4 individual wells 124. However, the processor 130 may be configured to communicate with any suitable number of wells, such as 1, 2, 3, 5, 6, 7, 8, 9, or 10 individual wells 122.

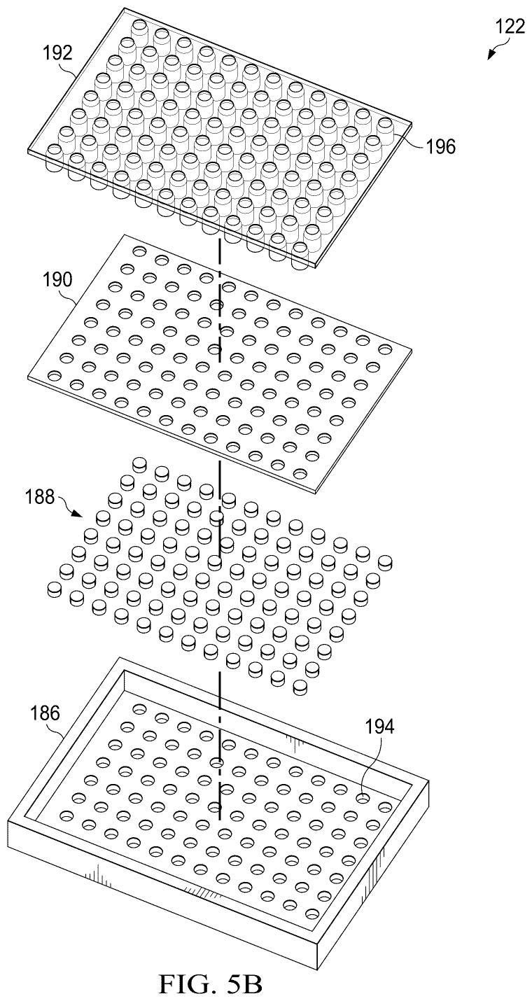

FIGS. 5A and 5B illustrate another embodiment of a multi-well plate 122, according to one or more embodiments of the present disclosure. FIG. 5A illustrates an assembled view of the multi-well plate 122 and FIG. 5B illustrates an exploded view of the multi-well plate 122. In the illustrated embodiment, the multi-well plate 122 is a 96-well plate having an 8×12 array of individual wells 124. The multi-well plate 122 in the illustrated embodiment includes four parts: a frame 186, bottoms 188 for each individual well 124, an electrical plate 190, and a well plate 192. The frame 186 includes holes 192 for fitting the bottoms 188 of the individual wells 124. The bottoms 188 may include one or more electrical probes for contacting the organoid, as described in more detail below. The well plate 192 includes cylinders that form the sides 196 of the individual wells 124 that couple to the bottoms 188 within the holes 194 of the frame 186. The electrical plate 190 is configured to fit around the individual wells 124. The electrical plate 190 may include one or more electrical leads 128 (shown in FIG. 4) for coupling each well 124 to one or more processor 130 (shown in FIG. 4). The electrical leads 128 may be electrically coupled to the probes in the bottoms 188 of the multi-well plate 122. In some embodiments, the one or more processors 130 may also be disposed on or within the electrical plate 190.

In some embodiments, the electrical plate 190 and/or the bottoms 188 of the wells 122 may be formed of a thin-film polymer. For example, in some embodiments, the electrical plate 190 and/or the bottoms 188 may be formed using polyimide thin film. In some embodiments, the thickness of the thin-film polymer may be less than or equal to 10 micrometers. The thin-film polymer may be optically transparent so that optical imaging can be used to image the organoids in the multi-well plate 122. In other words, the thin-film polymer may be transparent in an optical image of the multi-well plate 122. In some embodiments, the electrodes may be formed using a material that optimizes the signal-to-noise ration (SNR). For example, in some embodiments, the electrodes may be formed from platinum, iridium, or a conductive polymer.

In some embodiments, the number and size of the electrical leads 128 may be minimized to decrease the percentage of surface area that is blocked by the traces 128 during imaging. For example, in some embodiments, the electrical leads 128 may have a diameter of approximately 3 micrometers. In some embodiments, there may be only one lead 128 for each individual well 124.

In some embodiments, the multi-well plate 122 can be fabricated through commercial-grade microfabrication techniques that are known in the art to improve the repeatability, scalability, and cost-effectiveness of the multi-well plate.

Returning to FIG. 1, the mini-bioreactor chamber 102 may include one or more single-unit bioreactors 110. Each single-unit bioreactor 110 may be a closed-loop system that maintains a suitable environment for organoids to live. Thus, each single-unit bioreactor 110 may support a single experiment. Each single-unit bioreactor 110 may be disposed in an individual well 124 of the multi-well plate 122.

The single-unit bioreactors 110 may include an organoid basket 132 that is shaped and sized to hold an individual organoid 134. The organoid basket 132 may be covered by an organoid enclosure or top 136. The organoid enclosure 136 may be insulated to prevent heat from entering or exiting the single-unit bioreactor 110.

In some embodiments, multiple single-unit bioreactors 110 may be connected to each other through a local area network (e.g., the internet or Wi-Fi) so that multimodal experiments can be performed. In a multimodal experiment, organoids with different biological functions in different single-unit bioreactors 110 may be able to communicate with each other.

In some embodiments, each single-unit bioreactor 110 may include one or more sensors. Each sensor may be configured to measure a different parameter. For example, in some embodiments, each single-unit bioreactor 110 may include one or more of an oxygen sensor, a carbon dioxide sensor, a pH sensor, a temperature sensor, a glucose sensor, a metabolites sensor, a neurotransmitter sensor, a neuro signals sensor, or electrochemical impedance spectroscopy (EIS) 116.

The oxygen sensor, carbon dioxide sensor, pH sensor, temperature sensor, glucose sensor, metabolites sensor, and/or neurotransmitter sensor may be considered physical or chemical sensors 112. The oxygen sensor, carbon dioxide sensor, pH sensor, temperature sensor, and/or glucose sensor may detect critical parameters that are important for organoid's survival. The metabolite sensors and/or neurotransmitter sensors may detect organoid's metabolic activities, gene expression phenotypes, physiological, and/or pathological responses.

The neuro signal sensor may be a microelectrode array (MEA) 114 that is fabricated on a flexible and stretchable substrate. The MEA may have a teardrop configuration, which may be able to stretch to accommodate an organoid in different sizes and hang the organoid down into the media solution. The MEA may also be able to deliver an electric field to the organoid within the single-unit bioreactor 110.

In some embodiments, for each single-unit bioreactor 110, there may be two MEA designs. The first MEA design may be for surface recording and activation. The second MEA design may be for sub-surface recording and activation. The first MEA design may be disposed on the top of the organoid and the second MEA design may be disposed on the bottom of the organoid. In other embodiments, there is only one MEA design that contacts the bottom of the organoid. The EIS 116 may be a tool that monitors the histology of organoids and their overall health in real-time. In some embodiment, the single-unit bioreactor 110 may include a wave guide that directs optical light to the organoid 134 so that the response can be analyzed.

The fluid handling system 104 may be configured to deliver fluids to and/or remove fluids from each single-unit bioreactor 110. For example, the fluid handling system 104 may deliver liquid media, which contains nutrients for the organoids to survive, and oxygen (O₂) gas into each single-unit bioreactor 110 via an injection port 118. In some embodiments, the O₂ gas may include other gases as well such as, for example, nitrogen gas. Moreover, the fluid handling system 104 may remove metabolic waste from each single-unit bioreactor 110 via a waste drain 120. In some embodiments, the fluid handling system 104 may pump the metabolic waste out from each single-unit bioreactor 110. In some embodiments, The influx of nutrients and O₂ and the discharging of the metabolic waste by the fluid handling system 104 is designed to mimic an in vivo environment. In some embodiments, the temperature of the liquid media may be maintained at 37° C. This may be designed to mimic body temperature, which is also 37° C.

FIG. 6 illustrates a flow chart of the layers of the single-unit bioreactor 110, according to one or more embodiments. In some embodiments, the single-unit bioreactor 110 may include three fluid layers. The first or top layer 138 may receive liquid media and O₂ that is delivered to the single-unit bioreactor 110. In some embodiments, the single-unit bioreactor 110 may include one injection port 118 (as shown in FIG. 1) or, in other embodiments, may include multiple injections ports 118 (as shown in FIG. 6). In this particular embodiment, the single-unit bioreactor 110 includes separate injection ports 118 for each fluid: a liquid media injection port 140 and two O₂ gas injection ports 142. The top layer 138 may include one liquid media injection port 140 and one O₂ gas injection port 142.

The second or middle layer 144 of the single-unit bioreactor 110 may include the organoid 134. The middle layer 144 may receive liquid media and O₂ from the top layer 138 for cultivating the organoid. A second O₂ gas injection port 146 may allow O₂ gas to be delivered to the middle layer 144.

The third or bottom layer 148 of the single-unit bioreactor 110 may receive and/or collect waste from the middle layer 144 and/or top layer 138. The waste may be liquid or gaseous and may be produced by the organoid. The waste may also include excess fluid injected, such as excess liquid media and/or O₂ gas. A waste drain 120 may remove waste from the bottom layer 148.

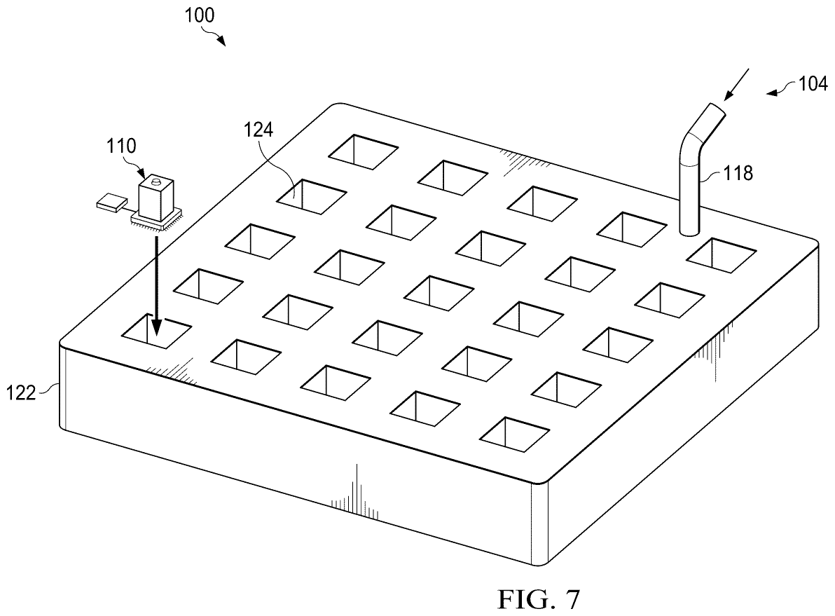

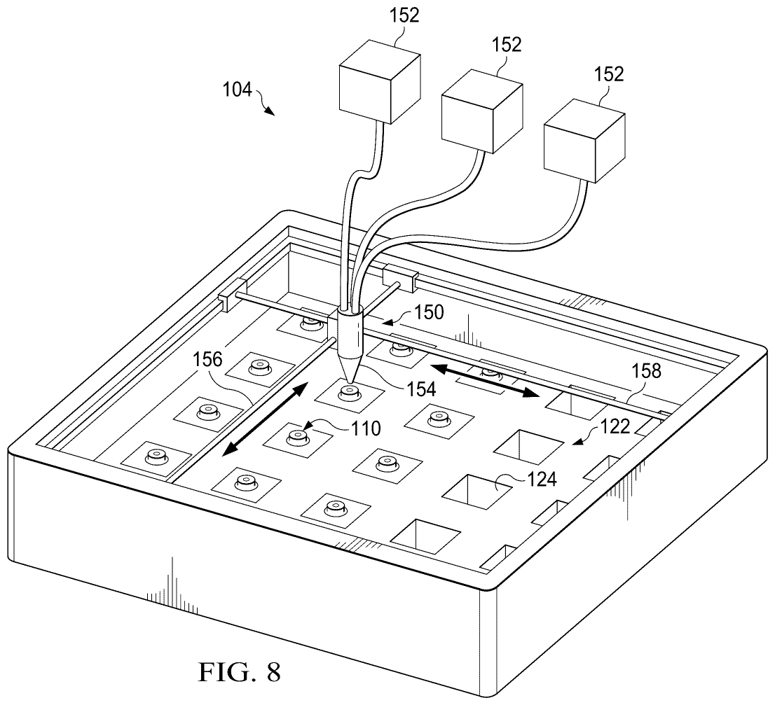

FIG. 7 illustrates a fluid handling system 104 and a multi-well plate 122, according to one or more embodiments of the present disclosure. FIG. 8 illustrates a fluid handling system 104 including a robotic dispenser 150 and a multi-well plate 122, according to one or more embodiments of the present disclosure. The fluid handling system 104 may include a robotic dispenser 150 that delivers fluids into the bioreactor chamber 102 via the injection port 118 (shown in FIGS. 1 and 7). The robotic dispenser may be controlled by the computer system 106, as described in more detail below. The robotic dispenser 150 may be programmable for injecting an accurate amount of a fluid into each single-unit bioreactor 110.

In some embodiments, the robotic dispenser may also be configured to deliver liquid media and/or O₂ gas to each single-unit bioreactor 110. In some embodiments, the robotic dispenser may also be configured to remove waste from each single unit bioreactor 110. In some embodiments, the robotic dispenser system may deliver drugs to the organoids in the single-unit bioreactors 110 for drug discovery and drug screening applications.

The fluid handling system 104 may include one or more holding chambers 152 for holding one or more fluids for injecting into the bioreactor chamber 102. For example, there may be separate holding chambers 152 for liquid media, for O₂ gas, and/or for drugs. A syringe, pipet, or dispensing tip 154 may be connected to each of the holding chambers 152. The dispensing tip 154 may dispense a known or controllable quantity of each fluid into a particular single-unit well 110. The dispensing tip 154 may be moveable across the top of the multi-well plate 122 so that it can deliver one or more of the fluids in the holding chambers 152 to each single-unit bioreactor 110. The dispensing tip 154 may be connected to a first conveyance system 156 and a second conveyance system 158. The first conveyance system 156 may move the dispensing tip 154 along a first axis. The second conveyance system 158 may move the dispensing tip 154 along a second axis. In some embodiments, the first axis and the second axis may be perpendicular. The computer system 106 may control the robotic dispenser 150 by controlling the conveyance systems 156, 158 to control movement of the dispensing tip 154 and/or controlling the dispensing tip 154 to dispense a certain amount of a certain fluid into a certain single-unit bioreactor 110.

The computer system 106 of the mini-bioreactor system 100 may include any suitable components. The computer system 106 may include one or more processors including, for example, central processing units, multi-core processors, microprocessors, microcontrollers, digital signal processors, field programmable gate arrays (FPGAs), application specific integrated circuits (ASICs), graphics processing units (GPUs) and/or the like. The computing device 106 may be implemented as a stand-alone subsystem, as a board added to a computing device, and/or as a virtual machine.

The computer system 106 may also include a memory that may be used to store software executed by computing device 106 and/or one or more data structures used during operation of computing device 106. The memory may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

The processor and/or the memory may be arranged in any suitable physical arrangement. In some embodiments, the processor and/or the memory are implemented on the same board, in the same package (e.g., system-in-package), on the same chip (e.g., system-on-chip), and/or the like. In some embodiments, the processor and/or the memory include distributed, virtualized, and/or containerized computing resources. Consistent with such embodiments, the processor and/or the memory may be located in one or more data centers and/or cloud computing facilities.

In some examples, the memory may include non-transitory, tangible, machine readable media that includes executable code that when run by one or more processors may cause the one or more processors to perform the methods described in further detail herein.

In some embodiments, the memory of the computer system 106 may include executable instructions or programs for a spike detection model that can be executed by a processor of the computer system 106. The spike detection model may receive measurements indicating the detection of action potential by one or more sensors 112 generated at the organoids. Additionally, the memory of the computation system 106 may include executable instructions or programs for impedance spectroscopy that can be executed by the processor of the computer system 106. The impedance spectroscopy instructions/programs may measure impedance of a single organoid.

Moreover, the computation system 106 may include one or more artificial intelligence (AI) models. An AI model may be trained to predict physical, chemical, or electrical stimuli for manipulating gene expressions of one or more of the organoids in the bioreactor chamber 102. In some embodiments, an AI model may be trained to detect and analyze neuro signal patterns and generate biological responses based thereon. In some embodiments, an AI model may be trained to identify lead compounds for drug discovery applications. In some embodiments, an AI model may be trained to identify drug toxicity and adverse effects of potential drug targets. In some embodiments, an AI model may be trained to identify drug interactions. In some embodiments, an AI model may be trained to screen for effective drugs for a certain disease. In some embodiments, an AI model may be trained to create a personalized treatment protocol for one or more diseases.

The memory of the computer system 106 may also include instructions or programs for an Internet of Things (IoT) model that can be executed by a processor of the computer system 106. The IoT model may provide global collaboration of various components of the mini-bioreactor system 100 at any point in time. The IoT model may provide real-time telemetry and closed-loop monitoring of one or more components of the mini-bioreactor system 100. The IoT model may observe data collected by one or more sensors in the bioreactor chamber 102. The IoT model may instruct the processor and/or memory computer system 106 to take immediate action based on the data.

In some embodiments, the IoT model may allow for interaction between organoids. For example, the IoT model may allow organoids in the system to communicate with each other through wireless communication such as the internet.

In some embodiments, the computer system 106 may control each single-unit bioreactor 110 so that experiments can be performed on the organoids 154 therein. For example, the computer system 106 may control, via electrical signals, the microelectrode array 160 to apply an electrical stimulus to the organoid 134. The computer system 106 may then receive electrical signals from the microelectrode array 160 and/or the sensors 112, 116. The electrical signals may represent data collected by the MEA 160 and/or the sensors 112, 116. The computer system 106 may analyze this data.

In some embodiments, experiments may be performed on one single-unit bioreactor 110. In some embodiments, experiments may be performed on a group of single-unit bioreactors 110 in the multi-well plate 122 such as, for example, a row or column of single-unit bioreactors 110. In other embodiments, experiments may be performed on all of the single-unit bioreactors 110 in the multi-well plate 122.

In some embodiments, the computer system 106 is spaced from the bioreactor chamber 102 and, in particular embodiments, may be located in another room or building from the bioreactor chamber 102. Thus, the computer system 106 can receive data and initiate actions remotely.

In some embodiments, the computer system 106 may also include various data security and privacy systems. The data security and privacy systems may be robust to preserve integrity of data and prevent hacking. The data security and privacy systems may use any suitable devices, systems, and/or programs known in the art.

The GUI may allow users to access and operate the mini-bioreactor system 100 on a desktop. In some embodiments, the GUI may allow users to access and operate the mini-bioreactor system 100 on a mobile device through an application. In some embodiments, the GUI may reduce the complexity of the software. In some embodiments, the GUI may enhance remote collaboration between laboratories. In some embodiments, the GUI may be disposed within the computer system 106. In some embodiments, the GUI may be implemented as a processor within the computer system 106.

Each single-unit bioreactor 110 may include an organoid basket 132 for holding and supporting an organoid 134. The organoid basket 132 may allow nutrients and gases to be transported across its structure so that they contact the organoid 134. The basket 132 may be configured to hold and support the 3D organoid 134 such that the organoid 134 can live, grow, incubate, and/or develop. The basket 132 may allow experiments to be performed on the organoid 134.

In some embodiments, the organoid basket 132 may be a hemispherical basket that is designed to host a spherical-shaped or rounded organoid 134. Thus, the hemispherical organoid basket 132 may support the organoid 134 on the bottom and on the sides. However, the basket 132 may be any suitable shape for support the 3D shape of the organoid 132. For example, in some embodiments, the basket 132 may be a cylinder, a cube, a rectangular prism, a hexagonal prism, conical, or any other suitable shape.

In some embodiments, the basket 132 may be perforated so that fluids such as liquid media, O₂ gas, or other nutrients can contact the organoid 134 and/or so that waste can move away from the organoid 134. The hemispherical basket 132 may support the round shape of organoid 134 over an extended period of time. In some embodiments, the organoid basket 132 may be sized on a millimeter-scale.

In some embodiments, the organoid basket 132 may be made of a 3D-printable material. In some embodiments, the basket 132 may be rigid. The organoid basket 132 may be assembled onto the microelectrode arrays 160 such that the arrays 160 are readily available for insertion when an organoid 134 is dropped on.

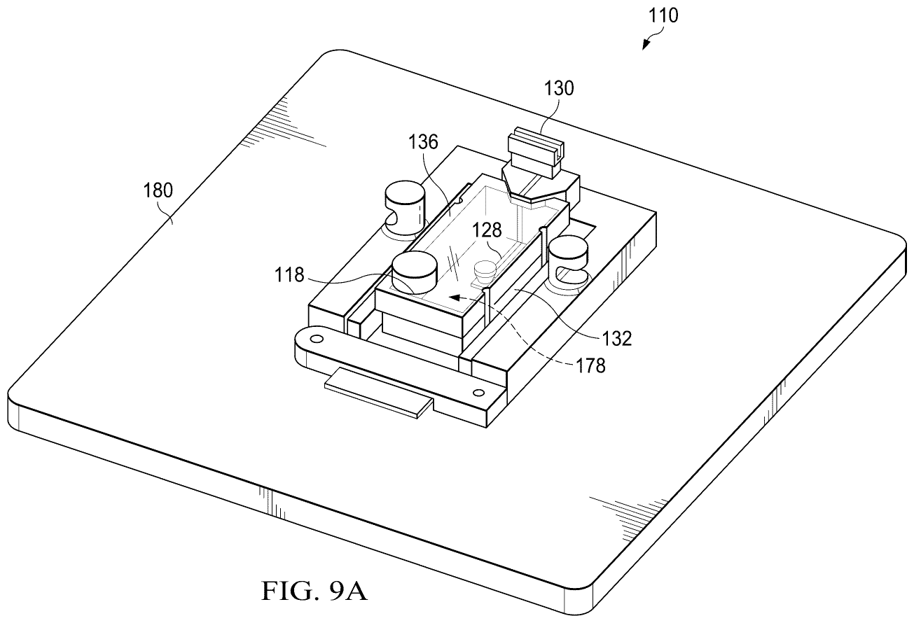

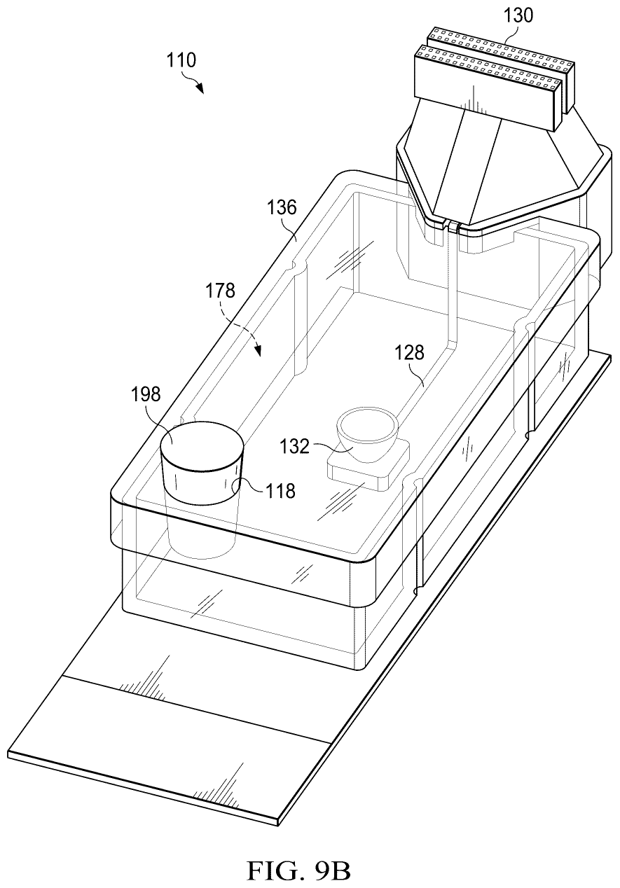

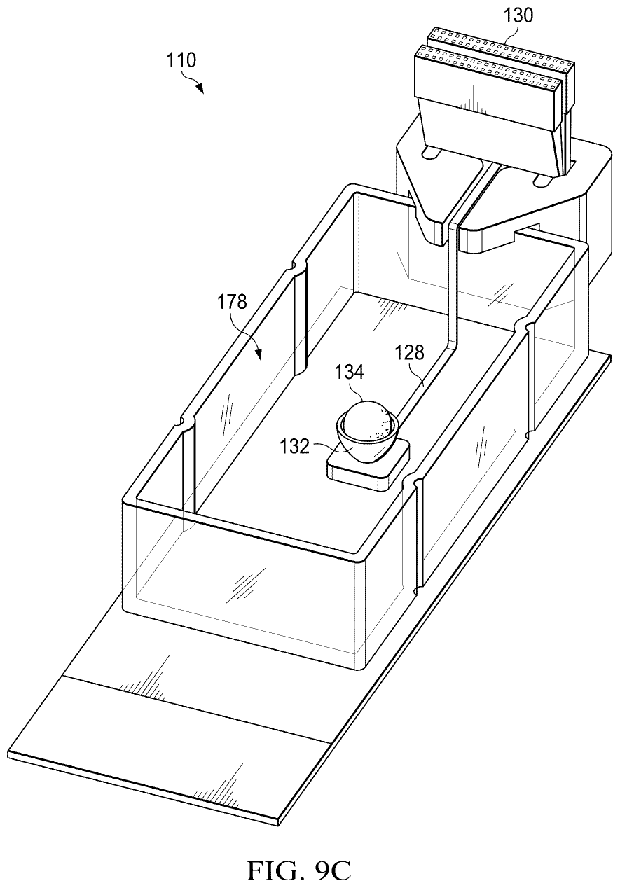

FIGS. 9A-9C illustrate an embodiment of a single-unit bioreactor 112 including a hemispherical organoid basket 132, according to one or more embodiments of the present disclosure. FIG. 9A illustrates the assembled single-unit bioreactor 112. FIG. 9B illustrates a zoomed-in view of the chamber 178 of the single-unit bioreactor of FIG. 9A. FIG. 9C illustrates a zoomed-in view of the chamber 178 of the single-unit bioreactor 112 of FIG. 9A with the organoid enclosure 136 removed and an organoid 134 in the basket 132.

The hemispherical basket 132 of the single-unit bioreactor 112 may be disposed in a media chamber 178. The media chamber 178 may be disposed on a stage 180. The stage 180 may provide stability for the media chamber 178, may ease the connection/disconnection of signal transmission cables, and/or may keep grounding wires in place. Electrical leads 128 may extend from the basket 132 to a processor 130. An organoid enclosure 136 may be disposed over the basket 132 to cover the basket 132 and media chamber 178. The organoid enclosure 136 may have an injection port 118 for receiving fluids such as liquid media and O₂ gas. A cover, plug, or stopper 198 may be inserted into the injection port 118 to close and/or seal the chamber 178.

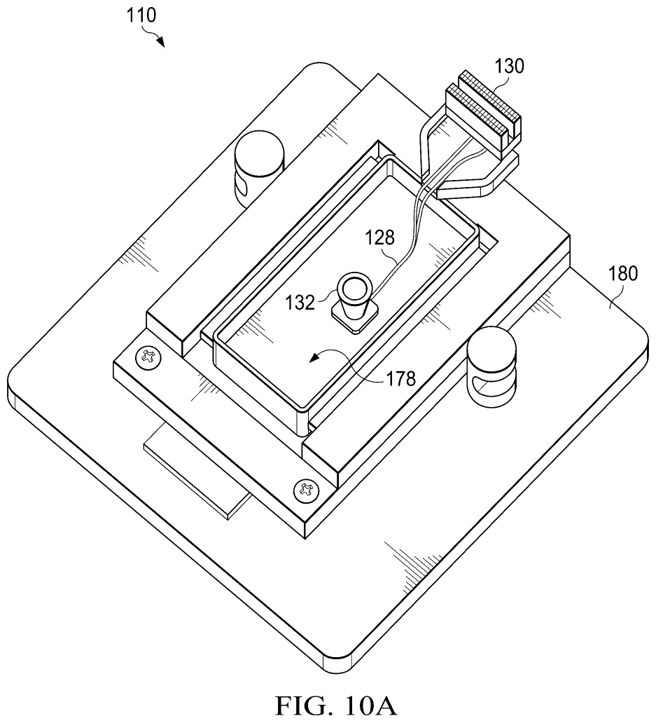

FIGS. 10A-10B illustrate another embodiment of the single-unit bioreactor 110, according to one or more embodiments of the present disclosure. The embodiment in FIGS. 10A-10B may be similar and/or identical to the embodiment illustrated in FIGS. 9A-9C. The single-unit bioreactor 110 includes a generally hemispherical basket 132 disposed in a media chamber 178. In some embodiments, the basket 132 may be generally conical. The media chamber 178 may be disposed on a stage 180. Electrical leads 128 may extend from the basket 132 to a processor 130. An organoid enclosure 136 may be disposed over the basket 132 to cover the basket 132 and media chamber 178. The organoid enclosure 136 may have an injection port 118 for receiving fluids such as liquid media and O₂ gas.

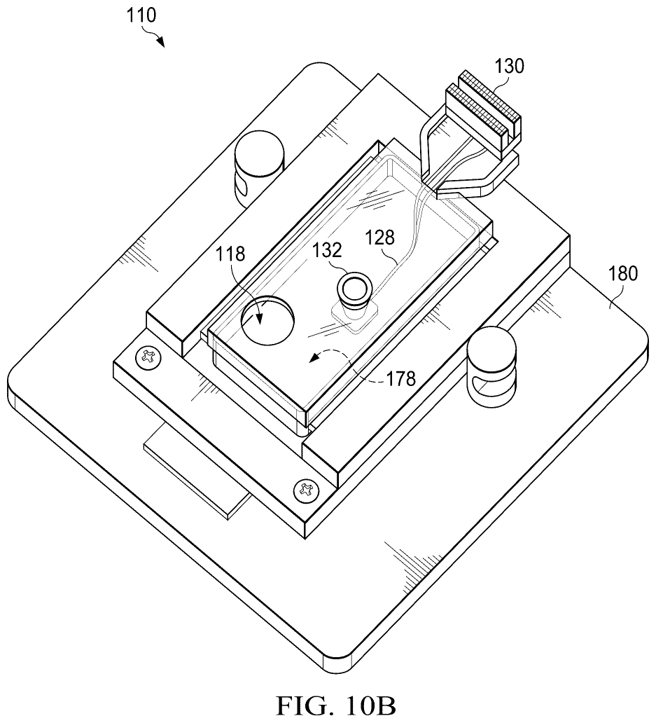

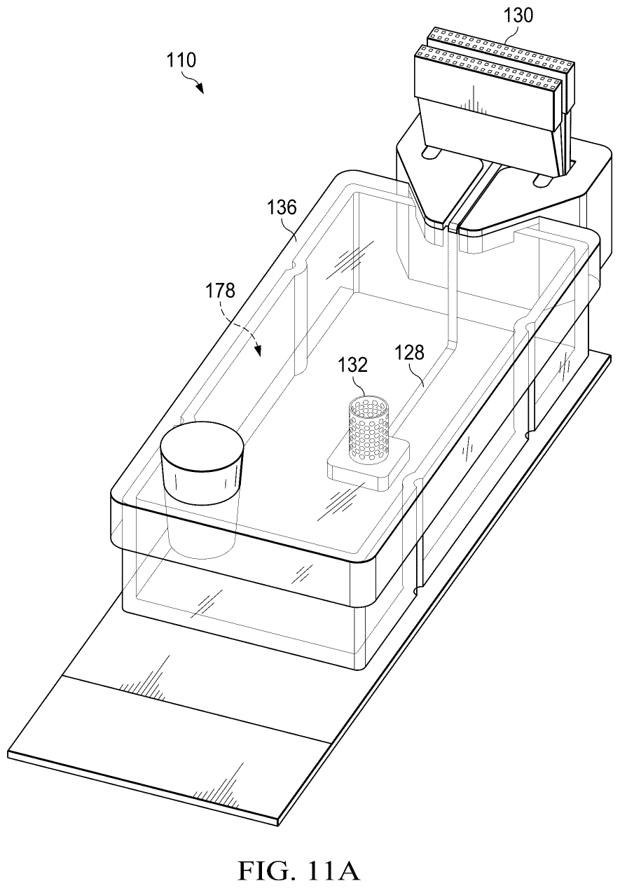

FIGS. 11A-11B illustrate another embodiment of a single-unit bioreactor 112 having a cylindrical basket 132, according to one or more embodiments of the present disclosure. FIG. 11A illustrates a perspective side view of the single-unit bioreactor 112 and FIG. 11B illustrates a perspective top view of the single-unit bioreactor 112 with the organoid enclosure 136 removed. The single-unit bioreactor 112 illustrated in FIGS. 11A-11B may be similar to the single-unit bioreactor 112 illustrated in FIGS. 9A-9C except that the single-unit bioreactor 112 illustrated in FIGS. 11A-11B includes a cylindrical basket 132 and the single-unit bioreactor 112 illustrated in FIGS. 9A-9C includes a hemispherical basket 132.

The cylindrical basket 132 of the single-unit bioreactor 112 may be disposed in a media chamber 178. The media chamber 178 may be disposed on a stage 180 (not shown in FIGS. 11A-11B). Electrical leads 128 may extend from the basket 132 to a processor 130. An organoid enclosure 136 may be disposed over the basket 132 to cover the basket 132 and media chamber 178. The organoid enclosure 136 may have an injection port 118 (not shown in FIGS. 11A-11B) for receiving fluids such as liquid media and O₂ gas. A cover, plug, or stopper 198 (not shown in FIGS. 11A-11B) may be inserted into the injection port 118 to close and/or seal the chamber 178.

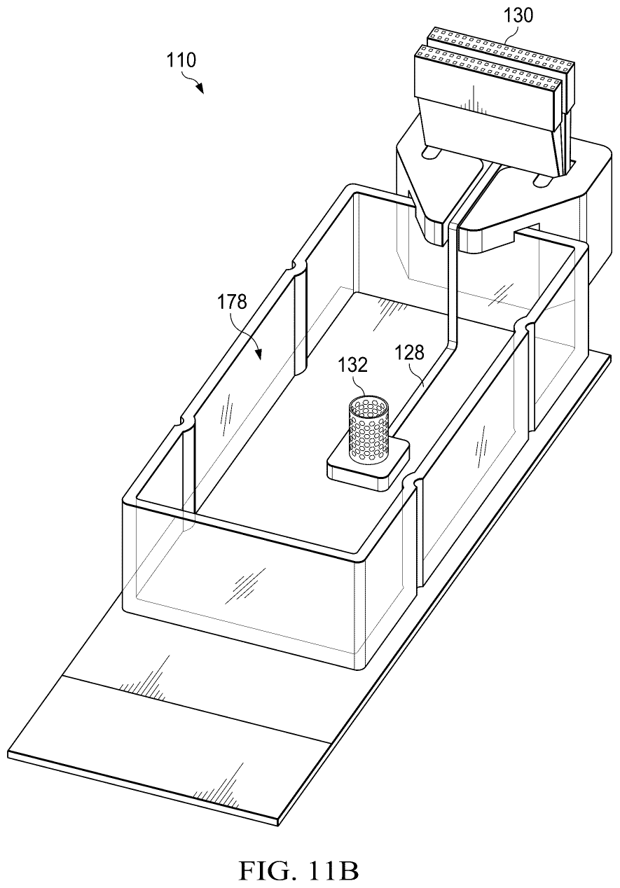

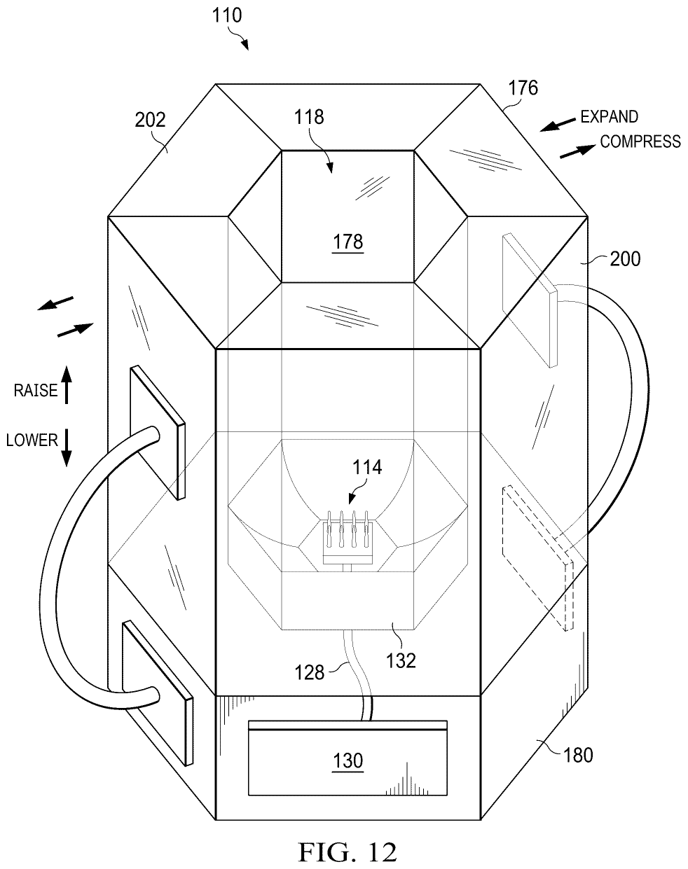

FIG. 12 illustrates another embodiment of a generally hemispherical organoid basket 132, according to one or more embodiments of the present disclosure. The organoid basket 132 may include a partially spherical bottom 162 and six curved sides 164 extending upward therefrom. Thus, the basket 132 may not be perfectly hemispherical, but may be at least partially spherical or curved.

The organoid basket 132 may be mounted in the middle of a media chamber 178. In some embodiments, the media chamber 178 may be mounted on a stage 180. The walls 200 of the chamber 178 may be extend vertically around the basket 132 and the tops 202 of the walls 200 may be bent inward to create a roof or top over the chamber 178. There may be a space or opening between the folded tops 202 of the walls 200 that may function as an injection port 118. Fluids such as media liquid and O₂ gas may be delivered to the chamber 178 via this injection port 118. Electrical leads 128 may extend from the basket 132 to a processor 130.

In some embodiments, the tops 202 of the walls 200 may be initially disposed in a first, unfolded position. In the unfolded position, the tops 202 may be configured to extend upward or outward from the basket 132 so that an organoid 134 can be placed in the bottom 162 of the basket 132. Once the organoid 134 is placed in the basket 132, the tops 202 may be moved to a second, folded position. The tops 202 may be folded so that they extend inward over the basket 132 (as shown in FIG. 12). The tops 202 may be formed of a flexible material that allows them to be bent, moved, or deformed from the unfolded position to the folded position. In some embodiments, the tops 202 may be formed of material that may hold its shape or become rigid upon application of a stimulus. For example, the tops 202 may be formed of a conjugated polymer, such as a shape memory polymer, that holds a set shape when a stimulus, such as heat, is applied. In some embodiments, the tops 202 may be formed of a conjugated polymer that becomes rigid upon application of a voltage to prevent movement of the tops 202.

Any of the baskets shown in FIGS. 9A-12 may include an MEA 114 (shown in FIG. 12) that is configured to contact and/or protrude or extend into the organoid 132.

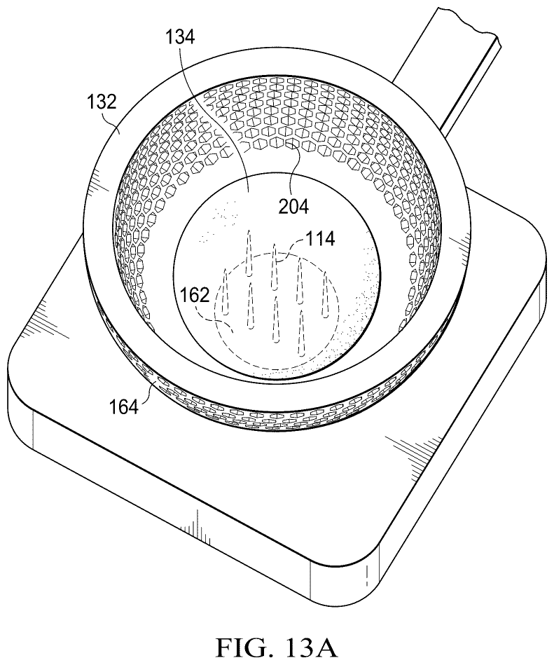

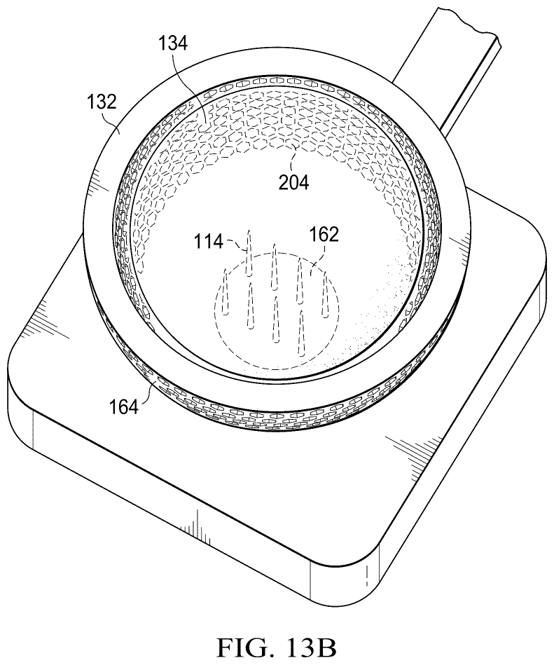

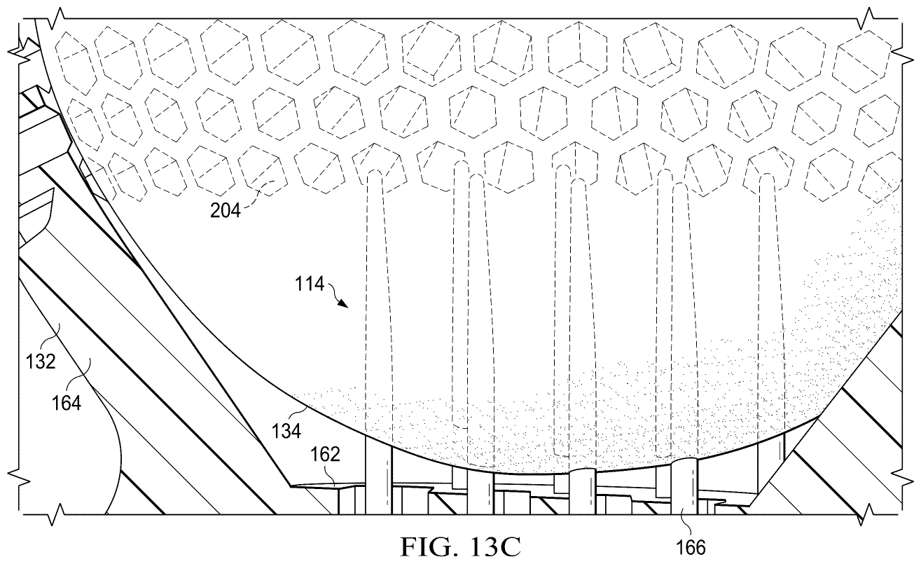

FIGS. 13A-13C illustrate a hemispherical organoid basket 132, according to one or more embodiments of the present disclosure. FIG. 13A illustrates the basket 132 with a smaller-sized organoid 134 disposed therein and FIG. 13B illustrates the basket 132 with a larger-sized organoid disposed therein. FIG. 13C illustrates a zoomed-in view of the MEA 114 of the basket 132 with the larger-sized organoid 134 disposed therein. The basket 132 illustrated in FIGS. 13A-13C may be the same or similar to the basket 132 illustrated in FIGS. 9A-9C.

As described above, the basket 132 is generally hemispherical and includes a plurality of perforations 204. The perforations 204 may extend along the sides 164 of the basket 132. In some embodiments, the bottom 162 of the basket 132 may not have perforations 204, but, in other embodiments, the bottom 162 may have perforations 204. The MEA 114 may be disposed on the bottom 162 of the basket 132. Thus, when the organoid 134 is placed in the basket 132, the electrode shanks 166 of the MEA 114 may protrude or extend into the organoid 134. When the organoid 134 is smaller-sized (as shown in FIG. 13A), the organoid 134 may only contact the bottom 164 of the basket 132. When the organoid 134 is larger-sized (as shown in FIGS. 13B-13C), the organoid 134 may contact or extend proximate to the sides 162 of the basket 132 in addition to contacting the bottom 164. The organoid 134 may be any suitable size. In some embodiments, a smaller-sized organoid 134 (as shown in FIG. 13A) may be, for example, approximately 2 millimeters in diameter. In some embodiments, a larger-sized organoid 134 (as shown in FIGS. 13B-13C) may be approximately 4 millimeters in diameter. However, the organoid 134 may be smaller than 2 millimeters in diameter, between 2-4 millimeters in diameter, or larger than 4 millimeters in diameter. For example, in some embodiments, the diameter may be in a range of 2-7 millimeters.

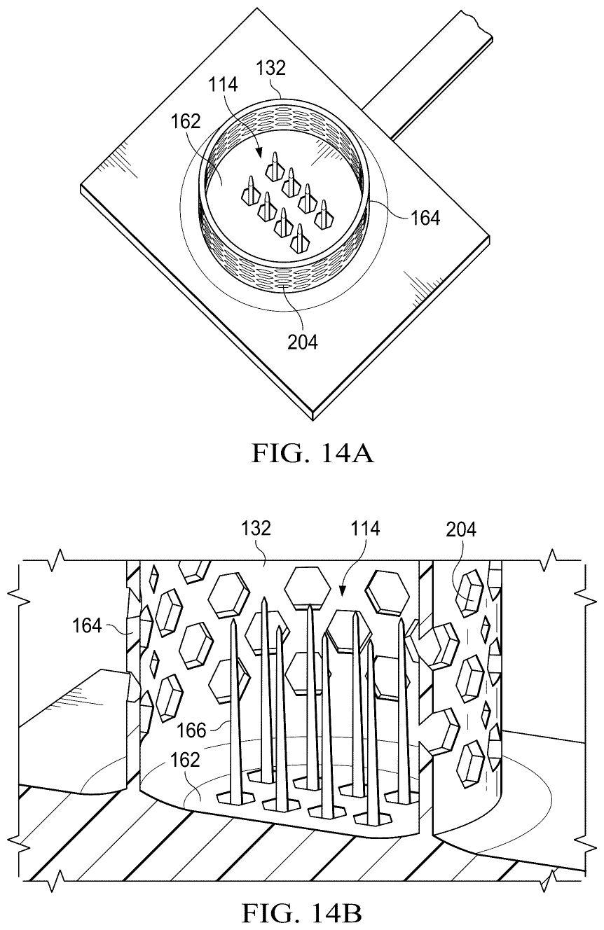

FIGS. 14A-14B illustrate a cylindrical basket 132, according to one or more embodiments of the present disclosure. FIG. 14A illustrates a perspective top view of the basket 132. FIG. 14B illustrates a zoomed-in view of the MEA 114 of the basket 132. The basket 132 illustrated in FIGS. 14A-14B may be the same or similar to the basket 132 illustrated in FIGS. 10A-10B.

As described above, the basket 132 is generally cylindrical and includes a plurality of perforations 204. The perforations 204 may extend along the sides 164 of the basket 132. In some embodiments, the bottom 162 of the basket 132 may not have perforations 204, but, in other embodiments, the bottom 162 may have perforations 204. The MEA 114 may be disposed on the bottom 162 of the basket 132. Thus, when the organoid 134 is placed in the basket 132, the electrode shanks 166 of the MEA 114 may protrude or extend into the organoid 134. When the organoid 134 is smaller-sized, the organoid 134 may only contact the bottom 164 of the basket 132. When the organoid 134 is larger-sized, the organoid 134 may contact or extend proximate to the sides 162 of the basket 132 in addition to contacting the bottom 164. The organoid 134 may be any suitable size. In some embodiments, a smaller-sized organoid 134 may be, for example, approximately 2 millimeters in diameter. In some embodiments, a larger-sized organoid 134 may be approximately 4 millimeters in diameter. However, the organoid 134 may be smaller than 2 millimeters in diameter, between 2-4 millimeters in diameter, or larger than 4 millimeters in diameter. For example, in some embodiments, the diameter may be in a range of 2-7 millimeters.

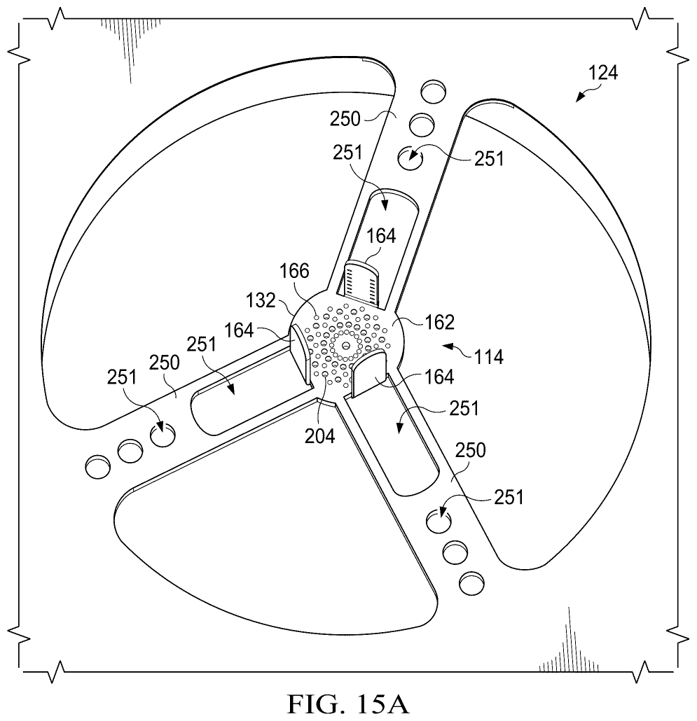

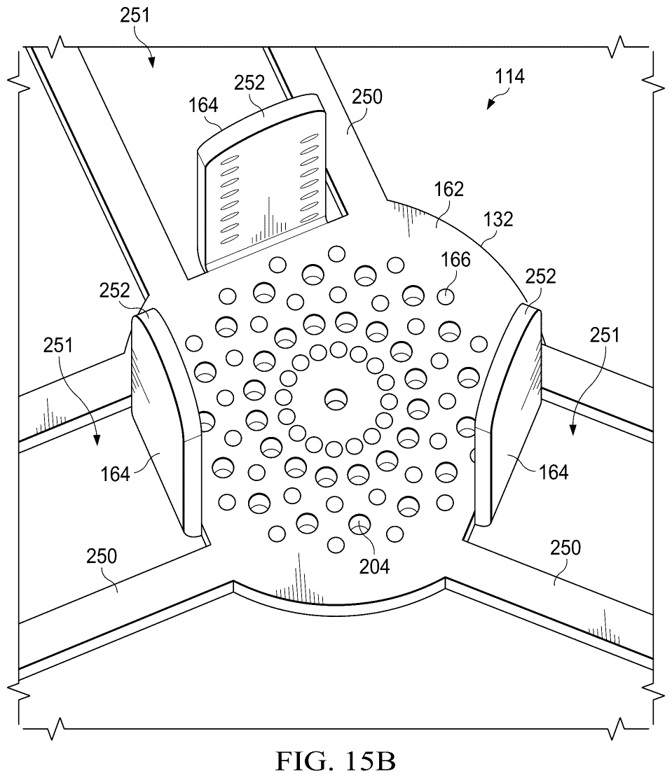

FIGS. 15A-15B illustrates another embodiment of an organoid basket 132, according to one or more embodiments of the present disclosure. FIG. 15A is a perspective view of the basket 132 and FIG. 15B is a zoomed-in view of the basket 132.

The basket 132 may include a bottom 162 and a plurality of sides 164. Each side 164 extends upward from the bottom 162. The basket 132 may include any suitable number of sides 164. In the illustrated embodiment, the basket 132 comprises three sides 164. However, in some embodiments, the basket 132 may comprise 1, 2, 4, 5, 6, 7, 8, 9, or 10 sides 164.

In some embodiments, the basket 132 may be configured to be inserted into an individual well 124 such as, for example, an individual well 124 of a 96-well plate. In some embodiments, the basket 132 may include supports or struts 250 that couple the basket 132 to the wall of the individual well 124. For example, the struts 250 may be coupled to the basket 132 at the bottom 162 around or at where the sides 164 are coupled to the bottom 162. In some embodiments, the struts 250 may hold the basket 132 so that it is submerged in a fluid (e.g. liquid media). The struts 250 may include one or more holes or openings 251 therethrough.

In some embodiments, the tops 252 of the sides 164 may be initially disposed in a first, unfolded position. In the unfolded position, the tops 252 may be configured to extend upward or outward from the basket 132 (as shown in FIGS. 15A-15B) so that an organoid 134 can be placed in the bottom 162 of the basket 132. Once the organoid 134 is placed in the basket 132, the tops 252 may be moved to a second, folded position. The sides 164 may be folded so that the tops 252 extend inward over the basket 132. The sides 164 and/or tops 252 thereof may be formed of a flexible material that allows them to be bent, moved, or deformed from the unfolded position to the folded position. In some embodiments, the sides 164 and/or the tops 252 thereof may be formed of material that may hold its shape or become rigid upon application of a stimulus. For example, the sides 164 and/or tops 252 thereof may be formed of a conjugated polymer, such as a shape memory polymer, that holds a set shape when a stimulus, such as heat, is applied. In some embodiments, the sides 164 and/or tops 252 thereof may be formed of a conjugated polymer that becomes rigid upon application of a voltage to prevent movement of the sides 164 and/or tops 252 thereof. When the sides 164 are in the folded position, the tops 252 may prevent the organoid 134 from floating upward and exiting the basket 132 and/or may ensure that the organoid 134 remains connected to the MEA 114.

The basket 132 may include a plurality of perforations 204. The perforations 204 may extend along the sides 164 of the basket 132. The bottom 162 of the basket 132 may also have perforations 204. The MEA 114 may be disposed on the bottom 162 of the basket 132. For example, the electrodes 166 of the MEA 114 may be dispersed between and/or around the perforations 204 on the bottom 162 of the basket 132. In some embodiments, the MEA 114 may also be disposed on one or more sides 164 of the basket 132. Thus, when the organoid 134 is placed in the basket 132, the electrodes 166 of the MEA 114 may contact and may extend partially or entirely through the organoid 134.

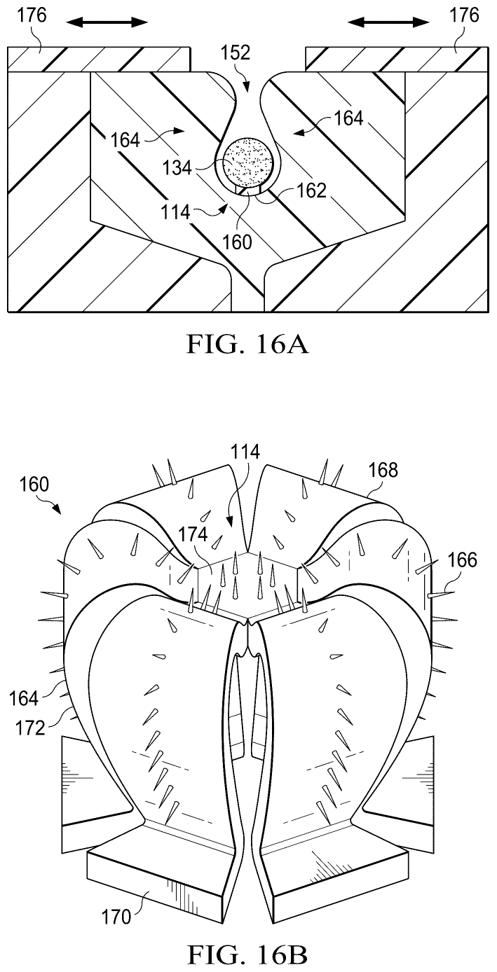

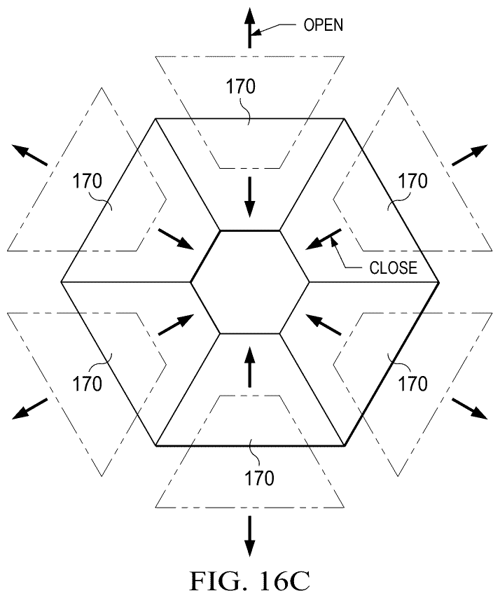

In some embodiments, the organoid basket 132 may not be rigid, but instead may be formed of a flexible MEA 114 that is folded around and supports the organoid 134. FIGS. 16A-16C illustrate a basket 132 formed of a flexible MEA 114, according to one or more embodiments of the present disclosure. FIG. 16A illustrates a cross-sectional view of an organoid basket 132 and FIG. 16B illustrates the MEA 114. FIG. 16C illustrates a top view of the opening/closing mechanism of the organoid basket 132.

In some embodiments, the organoid basket 132 may be formed of an MEA 114 with implantable microelectrodes that contact the organoid 134. In some embodiments, the bottom 162 of the MEA 114 includes electrodes 166 that penetrate the organoid 134 from the bottom once it is placed or dropped into the organoid basket 132. The sides of the MEA 114 may include electrodes 166 that penetrate the organoid 134 from the sides.

The microelectrode array 160 may be folded or arranged to encircle the organoid 134 from multiple (or all) directions such that the electrode shanks 166 penetrate radially into the organoid 134.

The microelectrode array 160 may be fabricated on a flexible and stretchable polymer substrate 168. One or more electrode shanks 166 may be formed perpendicular to the substrate 168. These electrode shanks 166 may establish stable and intimate contact with the organoid 134 compatible with changes within the operating environment. In various embodiments, any number of linear arrays of probes may be combined into a two-dimensional array to produce the MEA 160. For instance, the MEA 160 may include a 4×4 array including sixteen contact points that may serve as electrodes. However, the MEA 160 may be scalable such that, in various embodiments, the substrate 168 may include arrays and matrices of any dimensions. For example, in one embodiment, the substrate 168 may include a 2×10 array, while another embodiment may include a 6×7 array. By way of wires or thin-film conductive traces, a flexible ribbon cable coupled to the array 160 may provide electrical connectivity between the electrodes and the electronic circuits and a processor 130 (not shown in FIGS. 16A-16C) and/or components included in the computer system 106 (not shown in FIGS. 16A-16C). In some embodiments, a micro-LED or a waveguide can be incorporated on the substrate 168 or the electrode shanks 166.

While in various embodiments, the substrate 168 may be formed from a rigid material, the depicted substrate 168 is formed from a thin-film flexible material that permits the substrate 168 to deform. The deformation of the substrate 168 permits the substrate 168 to conform to the exterior surface of the organoid 134 to be monitored and/or stimulated by the electrodes of the substrate 168. Additionally, the deformation of the substrate 168 may enable the individual contact points serving as electrodes to move towards each other or away from each other as the underlying tissue expands and contracts, flexes, relaxes, or grows. The substrate 168 may include a thin-film based substrate made from polyimide, parylene, or silicone with embedded conductors (e.g., gold, platinum, etc.) that may electrically couple to the contact points serving as electrodes.

In addition to being flexible, the substrate 168 may be configured to include elastic spring-like properties such that the substrate 168 may deform in various ways to stretch, twist, buckle, and/or bend to withstand the tensile loads produced within the operating environment. That is, the substrate 168 may be provided with appropriate stiffness to enable reliable electrical contact and mechanical coupling with the biological tissues. This allows the substrate 168 to maintain robust conformal contact with the organoid 134.

In various embodiments, the substrate 168 may use surface tension to maintain continuous contact with the biological tissues. A fluid such as a hydrogel may be used to move the substrate 168 over the biological tissue. In addition, a flexible ribbon cable may enable constant electronic integration of the electrodes 166 with the electronic circuits and components within the processor 130 and/or computer system 106 for data collection and communication. In this way, the substrate 168 may be provided with structural reinforcements to yield a wide range of desired mechanical responses to changes within the operating environment while enabling predictable and reliable functionality of stimulating and/or sensing the organoid 134.

In some embodiments, the MEA 114 can be opened to allow the organoid 134 to be placed in the bottom 162 and can then be closed to insert the electrodes 166 into the organoid 134. The MEA 114 may also be reopened to release the organoid 134. For example, the MEA 114 may release the organoid 134 when an experiment is completed.

The MEA 114 is designed to branch out such that when the tips 170 of each branch 172 are pushed together, the center 174 of the MEA 114 stands up. In the standing configuration, the electrode shanks 166 at the center point upward and ready to penetrate the organoid 134. In some embodiments, the tips 170 of the branches 172 may form the base of the array 160 in the standing configuration (as shown in FIG. 16B).

Each tip/base 170 is mounted to the jaw 176 (shown in FIG. 16A) of a chuck, and the jaws 176 can be mechanically opened and closed in a radial direction.

FIG. 16C illustrates a top view of the MEA 114 (in particular, only the jaws 176 thereof are shown) in the open position (solid lines) and closed position (dashed lines), according to one or more embodiments of the present disclosure. In the open position, the jaws 176 are far apart so that the organoid 134 can be dropped onto the center 174 of the MEA 114. In some embodiments, the weight of the organoid 134 will pull the center 174 of the MEA 114 downward. As the center 174 of the array 160 is moved or pushed downward, the branches 172 of the MEA 114 encircle the organoid 134 such that the electrode shanks 166 along the branches 172 penetrate the organoid 134 as it moves downward. In this way, the configuration of the MEA 114 may be changed from standing (shown in FIG. 16B) to drooping (shown in FIG. 16A). The jaws 176 may then be moved to the closed position to move the MEA 114 into a closed position. In some embodiments, closing the jaws 176 may move the organoid 134 further downwards.

In some embodiments, liquid media is disposed below the jaws 176 of the chuck such that, in the closed position, the organoid 134 will be fully submerged in the liquid media. An experiment may be performed in the closed position when the organoid 134 is fully submerged in the liquid media. When the experiment is completed, the jaws 176 may be opened, so the organoid 134 can be removed. In some embodiments, the organoid 134 may be removed by pipetting. After removing the organoid 134, the MEA 114 may be cleaned and pulled back manually to its standing configuration.

The MEA 114 may also be any one of the microelectrode arrays described in U.S. Pat. Nos. 11,602,630, 8,954,144, and/or 9,248,269, the entirety of which are incorporated herein by reference.

The electrodes 166 of the MEA 114 may be placed in any suitable configuration about and through the organoid 134. In some embodiments, the MEA 114 may allow for the measurement of one or more properties that can be used to estimate the state of the organoid. For example, these properties may include one or more of cell diversities (e.g. by cell type) throughout the cell, cell fraction (which may be the percentage of the organoid's volume that is occupied by cells), cell types, spatial organization, or the pressure, state, or size of the inner necrotic core.

FIG. 17 illustrates an organoid 134 with a section removed, according to one or more embodiments of the present disclosure. The organoid 134 may be 3D and may be generally spherical, as illustrated. However, the organoid 134 may be any suitable 3D shape. The inner core of the organoid 134 may be a necrotic core 206, in which a high percentage of cells have died/are not viable. For example, in some embodiments, a majority (equal to or over 50%) of cells in the necrotic core 206 may not be viable. A healthy layer may be disposed around the necrotic core 206, in which a high percentage of cells are viable and a lower percentage of cells are not viable. For example, in some embodiments, a majority of the cells may be viable. This healthy layer may be referred to as the target region 208, because it may be more beneficial or advantageous to test the part of the organoid 134 with mostly healthy, live, or viable cells.

The organoid 134 may be any suitable size. For example, in the illustrated embodiment, the organoid 134 has a diameter of approximately 3 millimeters. Moreover, in this embodiment, the healthy layer or target region 208 is approximately 400 micrometers thick. However, the target region 208 may be any size.

The organoid 134 may be formed of any suitable type of cells. For example, the organoid may include neurons, cardiac cells, lung cells, kidney cells, or any other type of cells.

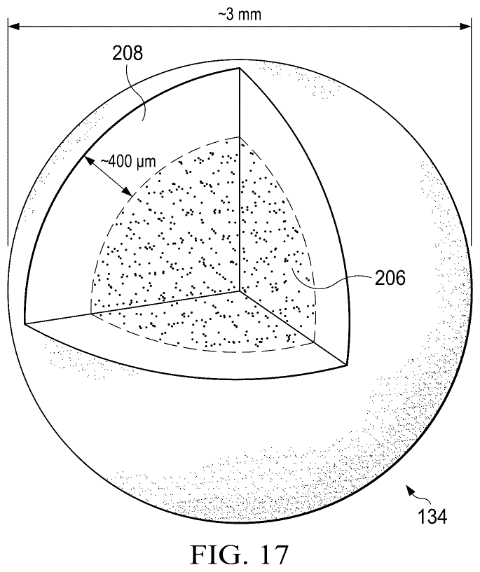

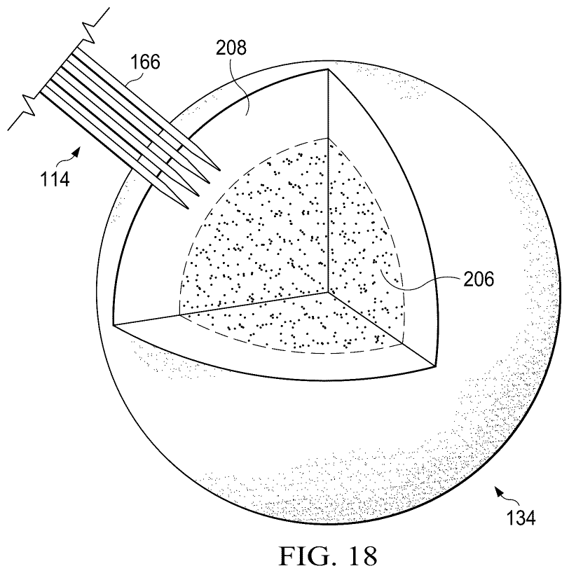

FIG. 18 illustrates an MEA 114 inserted into the target region 208, according to one or more embodiments of the present disclosure. In the illustrated embodiment, the electrodes 166 do not extend into the necrotic core 206. However, in other embodiments, the electrodes 166 may extend into the necrotic core 206 and/or may extend therethrough to the opposite side of the target region 208.

The MEA 114 may measure one or more properties of the organoid 134 by measuring the impedance through the organoid 134. In some embodiments, the MEA 114 may be configured to perform electrical impedance spectroscopy (EIS) of the organoid 134. In some embodiments, the MEA 114 may be configured to perform electrical impedance tomography (EIT) of the organoid 134.

In some embodiments, the MEA 114 may include penetrating electrodes 210 (as shown in FIG. 18). However, in other embodiments, the MEA 114 may include surface electrodes 212 (as described below). In some embodiments, the MEA 114 may include both penetrating electrodes 210 and surface electrodes 212.

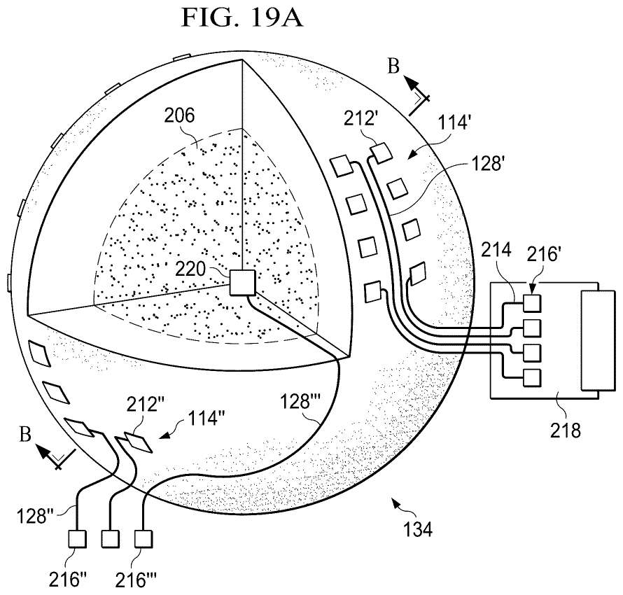

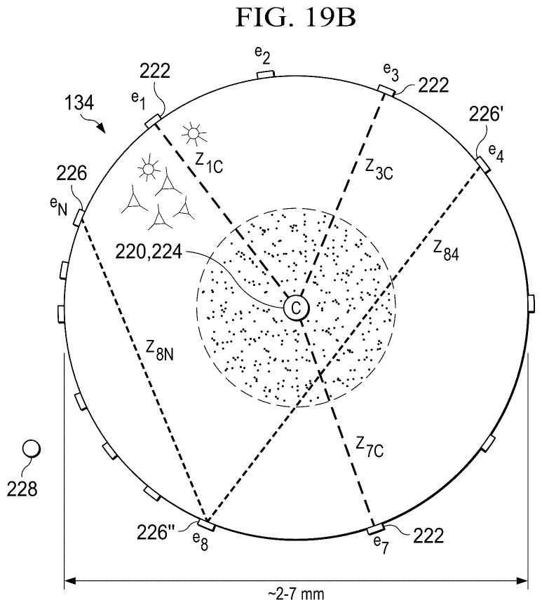

FIGS. 19A-19B illustrate an organoid 134 with at least one MEA 114 including a plurality of surface electrodes 212, according to one or more embodiments of the present disclosure. FIG. 19A is a perspective view of an organoid 134 with one or more MEAs 114 disposed on the surface thereof. FIG. 19B is a cross-sectional view of an organoid 134 with one or more MEAs 114 that may be taken, for example, through the B-B line shown in FIG. 19A.

A first MEA 114′ may include a plurality of surface electrodes 212′ and is positioned on a first region of the organoid 134. In some embodiments, the first MEA 114′ may include a plurality of electrical leads 128′ that connects each surface electrode 212′ with a bond pad segment 214. In some embodiments, a plurality of bond pads 216′ may be disposed on a printed circuit board (PCB) 218. The PCB 218 may be electrically coupled to a processor (DAQ) (not shown in FIGS. 19A-19B) for data acquisition and analysis. The DAQ may be the processor 130 or may be included in computer system 106 described above. In some embodiments, the first MEA 114′ may span a particular region of the organoid 134. Thus, the first MEA 114′ may not span a larger region of the organoid or the entire organoid 134. Thus, in some embodiments, multiple first MEAs 114′ may be disposed on different regions of the organoid 134 so that the organoid is fully covered.

In some embodiments, a second MEA 114″ may also include a plurality of surface electrodes 212″. Each surface electrode 212″ of the second MEA 114″ may be connected to a bond pad 216″ via an electrical lead 128″. In some embodiments, the bond pads 216″ of the second MEA 114″ may not be coupled to a PCB 218 or DAQ like the bond pads 216′ in the first MEA 114′. Thus, the second MEA 114″ may be disposed on a larger region of the organoid 134 than the first MEA 114′. For example, the second MEA 114″ may span one or more hemispheres of the organoid 134.

In some embodiments, a core electrode 220 disposed within the necrotic core 206 of the organoid 134. In some embodiments, the core electrode 220 may be a penetrating electrode 210. The core electrode 220 may be coupled to a bonding pad 216′″ that is exterior to the organoid 134 via an electrical lead 128′″. The core electrode 220 may optionally be used.

In some embodiments, there may be at least one first MEA 114′ and at least one second MEA 114″ disposed on the organoid 134. In some embodiments, there may only be at least one first MEA 114′ disposed on the organoid 134. In some embodiments, there may only be at least one second MEA 114″ disposed on the organoid 134. In some embodiments, a core electrode 220 is disposed within the core 208 of the organoid. In some embodiments, there is no core electrode 220.

One or more electrodes may interact to measure the impedance through the organoid 134, as shown in FIG. 19B. In some embodiments, two electrodes may be used to measure the impedance. For example, in some embodiments, a first electrode 222 may emit an electrical signal and a second electrode 224 may be configured to receive the emitted electrical signal from the first electrode 222. Thus, the first electrode 222 may be referred to as a working electrode and the second electrode 224 may be referred to as a sink or reference electrode. In some embodiments, one or more surface electrodes 210 may be working electrodes 222 and a core electrode 220 may be a reference electrode 224, as shown in the illustrated embodiment. However, in other embodiments, the core electrode 220 may be a working electrode 222 and/or one or more surface electrodes 210 may be reference electrodes 224.

In some embodiments, three electrodes may interact to measure the impedance through the organoid 134. For example, in some embodiments, a first electrode 226 at a first position and a second electrode 226′ at a second position may be configured to emit an electrical signal to a third electrode 226″ at a third position. Thus, the first electrode 226 and second electrode 226′ may be referred to as working electrodes and the third electrode 226″ may be referred to as a reference electrode. In some embodiment, the third electrode 226″ may be configured to emit an electrical signal to both the first electrode 226 and the second electrode 226′. Thus, the third electrode 226″ may be referred to as a working electrode and the first electrode 226 and second electrode 226′ may be referred to as reference electrodes. In some embodiments, a further reference electrode 228 may be spaced from the organoid 134. The further reference electrode 228 may be disposed within the single-unit bioreactor 110.

There are several design parameters that may be considered when designing the one or more MEAs 114 to use for measuring impedance through an organoid 134. In some embodiments, a three-electrode design may provide more accurate or precise impedance measurements compared to a two-electrode design. However, the three-electrode design may be more complex and more expensive than the two-electrode design. Moreover, the design may be chosen to optimize one or more parameters including, for example, inter-site spacing, site size, site material, strain relief, stretchability, flexibility, and transparency (which may be a function of the wavelength). In some embodiments, a goal of designing one or more MEAs 114 may be to optimize the estimation of the core region size using the least complex grid. This may be performed by selecting a combination of one or more of the above mentioned parameters. For example, in some embodiments, the resolution of the impedance may be increased when the inter-site spacing between electrodes is decreased.

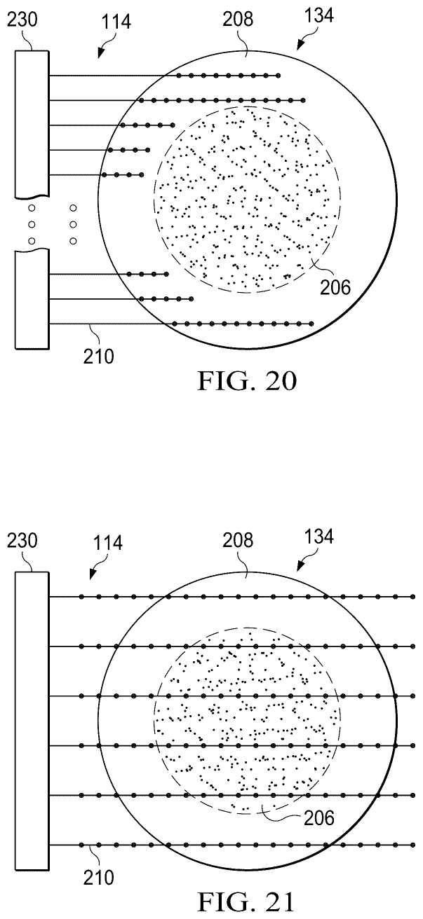

FIG. 20 illustrates a cross-sectional view of an embodiment of a MEA 114 including a plurality of penetrating electrodes 210 extending into an organoid 134, according to one or more embodiments of the present disclosure. In some embodiments, as in the illustrated embodiment, the penetrating electrodes 210 may extend into the target region 208 of the organoid 134 without extending into the necrotic core 206. Thus, in some embodiments, each penetrating electrode 210 may be a different length depending on where it is located relative to the core 206. Each penetrating electrode 210 may be coupled to a back-end of platform 230. In some embodiments, the MEA 114 may have a 2D line of penetrating electrodes 210 that extend into the organoid 134. In some embodiments, the MEA 114 may have a 3D area of penetrating electrodes 210 that extend into the organoid 134. The penetrating electrodes 210 may be regularly or irregularly spaced from each other. In some embodiments, the platform 230 may be coupled to the bottom of an organoid basket 132 (not shown in FIG. 20) so that the penetrating electrodes 210 penetrate the organoid 134 from the bottom.

FIG. 21 illustrates another embodiment of a MEA 114 including a plurality of penetrating electrodes 210 extending into an organoid 134, according to one or more embodiments of the present disclosure. Unlike the embodiment of the MEA 114 illustrated in FIG. 21, the penetrating electrodes 210 of the MEA 114 illustrated in FIG. 21 extend into and through the necrotic core 206 in addition to the target region 208. Thus, in some embodiments, the length of each penetrating electrode 210 may be approximately the same. Each penetrating electrode 210 may be coupled to a back-end of platform 230. In some embodiments, the MEA 114 may have a 2D line of penetrating electrodes 210 that extend into the organoid 134. In some embodiments, the MEA 114 may have a 3D area of penetrating electrodes 210 that extend into the organoid 134. In some embodiments, the MEA 114 may include a 1D point of one or more penetrating electrodes 210 that extend into the organoid 134. The penetrating electrodes 210 may be regularly or irregularly spaced from each other. In some embodiments, the platform 230 may be coupled to the bottom of an organoid basket 132 (not shown in FIG. 21) so that the penetrating electrodes 210 penetrate the organoid 134 from the bottom.

In some embodiments, a goal of designing the MEA 114 with penetrating electrodes 210 may be to optimize recording quality and probe complexity. For example, optimizing recording quality may include optimizing one or more of SNR, the number of cells (e.g., neurons) recorded, and/or the stability of the system. For example, in some embodiments, a goal may be to record from a maximum number of neurons while minimizing the number of electrode sites used. Moreover, in some embodiments, a goal may be to record coverage within a field or region of interest (e.g. a column or 2D line of penetrating electrodes 210) with a minimum number of electrode sites. Additionally, in some embodiments, a goal may be to match the design of an algorithm for analyzing the organoid (e.g. spike sorting) using the fewest amount of resources (e.g. minimizing electrode sites, electrical leads, and/or bond pads).

In some embodiments, the penetrating electrode 210 may be configured to measure a region of interest around the shank. For example, in some embodiments, the electrode 210 may measure a region of interest of approximately 250 micrometers around the shank. Thus, the region of interest for the electrode 210 may form a cylindrical volume around the electrode 210. In some embodiments, a goal of the experiment may to have a high percentage of coverage within that region of interest around the shank 210. For example, a goal may be to measure, record, or analyze a range of 50% to 100% of the volume of the region of interest. Additionally, in some embodiments, a goal may be to have a high coverage of the organoid 134. Thus, the goal is to have the regions of interest around the electrode 210 cover the entire organoid 134 or a sample of the entire organoid 134. For example, in some embodiments, a goal may be to cover a range of 50% to 90% of the total volume of the organoid 134. The coverage may be a function of the shape and/or size of the organoid 134.

In some embodiments, the MEA 114 may be configured to support and/or hold the 3D organoid 134. For example, when an MEA 114 includes one or more penetrating electrodes 210, the penetrating electrodes 210 may be configured to support and hold the organoid 134 so that it maintains a 3D shape. In another example, an MEA 114 with surface electrodes 212 may also be configured to support and hold the organoid 134 so that it maintains a 3D shape. In some embodiments, the surface electrodes 212 may be disposed on a mesh or structure that encircles and/or support at least a portion of the organoid 134.

Moreover, the MEA 114 may be designed to allow access to the surface of the organoid 134. For example, the MEA 114 may have openings, holes, or other spaces that allows fluids (e.g. liquid media) to flow through and contact the organoid 134. In some embodiments, one or more electrodes 166 (surface 212 or penetrating 210) may be disposed on a mesh structure. The mesh structure may be designed to minimize the amount of area that the electrodes 166 and/or mesh covers. In some embodiments, the MEA 114 may cover a range of 10 to 90% of the surface area of the organoid 134.

Additionally, each electrode 166 (surface 212 or penetrating 210) of any of the MEAs 114 described herein may be individually addressable or controllable. Thus, experiments may be performed by controlling each individual electrode 166 or by controlling a subset of the total number of electrodes 166 in the MEA 114.

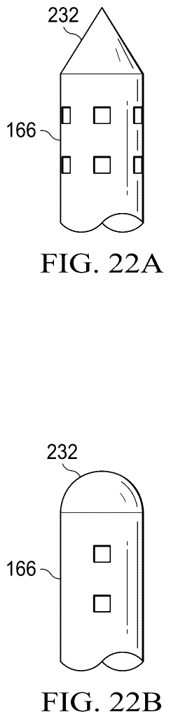

Any suitable electrode 166 may be used in the MEA 114 of any embodiments of the basket 132 described herein. FIGS. 22A-22B illustrate embodiments of an electrode shank 166, according to one or more embodiments of the present disclosure. FIG. 22A illustrates a shank 166 with a sharp tip 232 and FIG. 22B illustrates a shank 166 with a rounded tip 232.

In some embodiments, the electrode shanks 166 may be relatively flexible or, in other embodiments, the shanks 166 may be relatively stiff. In some embodiments, the shanks 166 may include one or more holes therethrough to promote cellular ingrowth and/or to minimize the obstruction of the organoid 134 with the shank 166.

In some embodiments, a goal of the shank 166 design may be to increase the coverage of recording while minimizing the damage to the organoid 134 during insertion of the shank 166. For example, the tip shape, insertion speed, and/or shank separation may be controlled. For example, in some embodiments, increasing the sharpness of the tip 232 and increasing the rate of speed of insertion may minimize the damage to the organoid 134. Moreover, in some embodiments, decreasing the sharpness of the tip 232 and decreasing the rate of speed of insertion may minimize damage to the organoid 134.

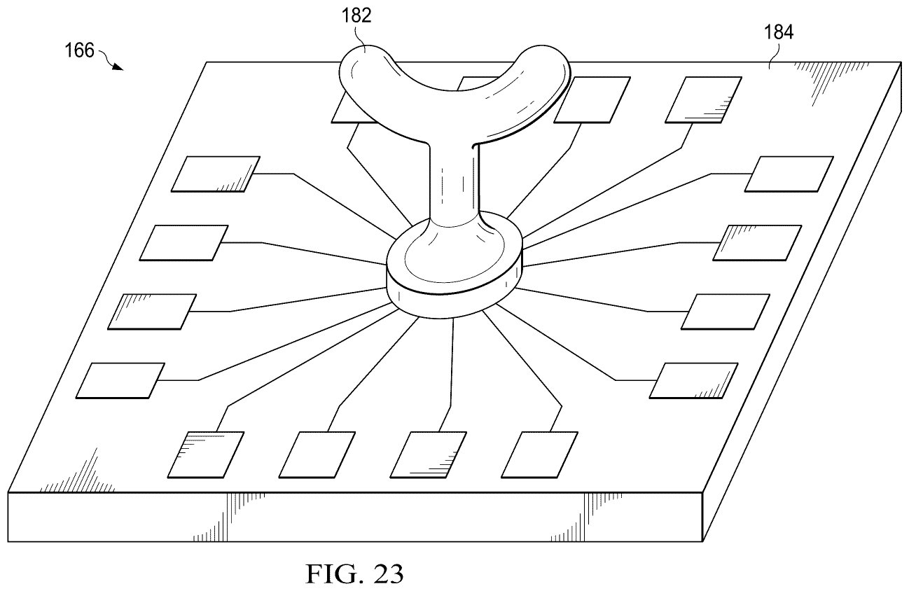

FIG. 23 illustrates an embodiment of an electrode shank 210, according to one or more embodiments of the present disclosure. The microelectrode shank 210 may include a receptor 182 coupled to a chip 184. The receptor 182 may be configured to contact the organoid 134.

However, any suitable electrode 166 design may be used in the MEA 114. For example, the MEA 114 may include electrode probes such as any of those described in U.S. Pat. Nos. 7,941,202, 8,195,267, 8,332,046, 8,224,417, 8,565,894, 8,688,231, 8,731,673, 8,870,857, 8,958,862, 8,958,890, 8,972,026, 9,008,747, 9,014,796, 9,289,142, and/or 8,948,843, the entirety of which are incorporated herein by reference.

The electrodes 166 may be inserted into the organoid 134 in any suitable way. For example, electrodes 166 disposed on the bottom of a basket 132 may penetrate an organoid 134 when it is dropped into the basket 132. In some embodiments, the electrodes 166 may be pressed into the organoid. However, when using thin or ultrathin electrodes 166, pressing the electrode 166 into the organoid 134 may cause the electrode 166 to bend or break.

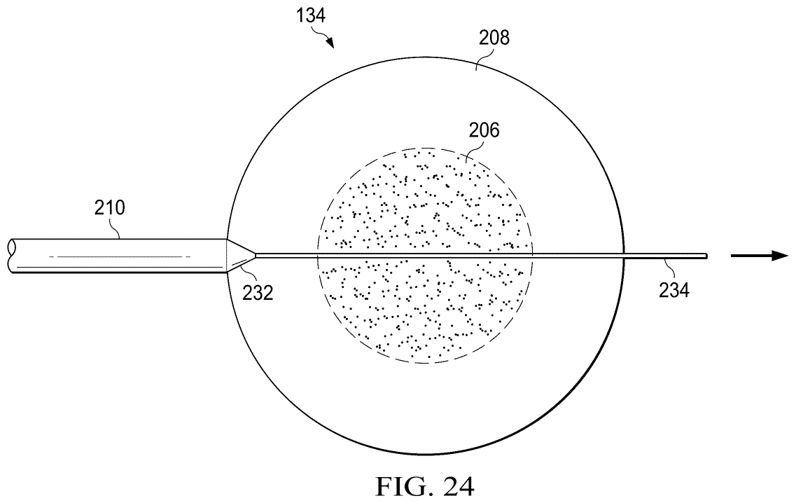

Thus, FIG. 24 illustrates a method of inserting a thin or ultrathin electrode 210 into an organoid 134, according to one or more embodiments of the present disclosure. In this embodiment, a line or wire 234 is coupled to the tip 232 of the penetrating electrode 210 on one end. The other end of the wire 234 is then inserted and threaded through the organoid 134 at the location where the electrode 210 is to be inserted. Once the wire 234 is threaded through the organoid 134, the wire 234 can be pulled to pull the electrode 210 into the organoid 134, as indicated by the arrow in FIG. 24. Once the electrode 210 is in place, the wire 234 can be removed, leaving the electrode 210 in place. Pulling the electrode 210 into the organoid 134 from the tip 232 rather than pushing the electrode 210 into the organoid 134 may prevent bending or breaking of the electrode 210 during insertion.

The wire 234 may be coupled to the electrode 210 in any suitable way. For example, in some embodiments, the wire 234 may be looped through an opening in the tip 232 of the electrode 210. In other embodiments, the wire 234 may hook onto an opening or cavity in the tip 232.

Although an electrode 210 with a relatively sharp tip 232 is illustrated in FIG. 24, any suitable electrode 210 may be inserted using this method. In some embodiments, this method of insertion may also be used for electrodes 210 that are not thin or ultrathin.

In some embodiments, a cannula or tube may also be inserted into the organoid 134 in the desired location. The electrode 210 may then be inserted into the opening of the tube. The tube may then be removed, leaving the electrode 210 in place within the organoid 134. The electrode 210 may include a flip anchor to hold it to the organoid 134 as the tube is removed. Thus, the flip anchor may hold the electrode 210 in the desired location.

In some embodiments, the penetrating electrode 210 may also include a coating over part or all of the probe 210. In some embodiments, the coating may improve electrical, mechanical, and/or biological characteristics of the probe 210 over time. The coating may be permanent or resorbable. For example, in some embodiments, the probe 210 may be coated with one or more bioactive coatings to decrease the damage to the cells (e.g. neurons) of the organoid 134 over time. In some embodiments, the one or more bioactive coatings may decrease the reactive response of the cells (e.g. neurons) of the organoid 134 over time. In some embodiments, the bioactive coating may include a hydrogel with an anti-inflammatory effect. In some embodiments, the probe 210 may be coated with one or more electrode coatings and/or one or more drug eluting coatings to affect the growth and/or development of the organoid 134.

In some embodiments, it may be beneficial to deliver nutrients or remove waste from the necrotic core 206 of the organoid 134. In some embodiments, by allowing nutrients to access the core 206 rather than just the outer layer 208 of the organoid 134 may allow a higher percentage of cells in the core 206 to live. This may improve analysis of the organoid 134 as a whole and may mirror the native biology better.

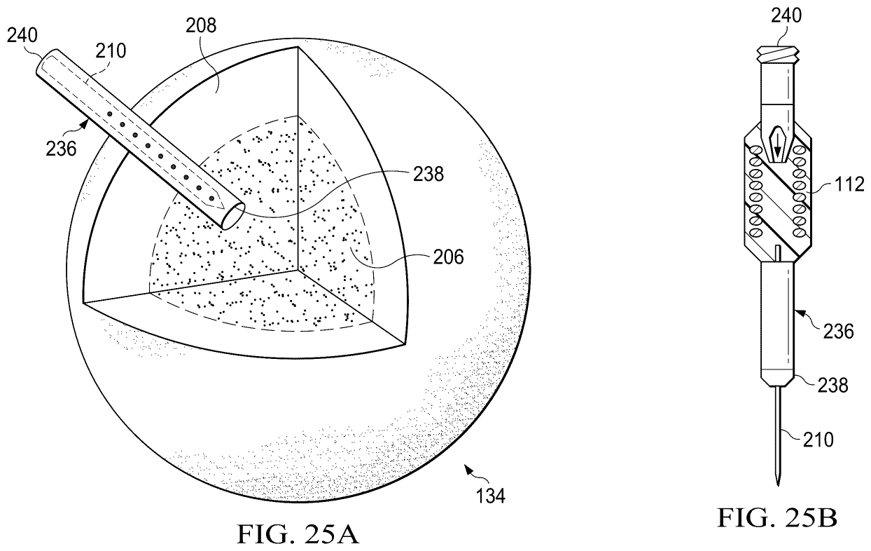

FIGS. 25A-25B illustrate an embodiment of a penetrating electrode 210 system for delivering nutrients to the necrotic core 206, according to one or more embodiments of the present disclosure. FIG. 25A illustrates the penetrating electrode 210 system inserted into an organoid 134 and FIG. 25B illustrates a detailed view of the penetrating electrode 210 system.

The penetrating electrode 210 may be coupled to a cannula 236. In some embodiments, the electrode 210 may be coupled to the outside of the cannula 236. In some embodiments, the electrode 210 may be coupled to the interior opening of the cannula 236. The cannula 236 and the electrode 210 coupled thereto may be inserted into the organoid 134 such that a first open end 238 of the cannula 236 is disposed in the necrotic core 206. In some embodiments, system may be inserted such that the electrode 210 couped to the cannula 236 is also at least partially disposed within the necrotic core 206. In some embodiments, the electrode 210 may be used to determine whether the first open end 238 of the cannula 236 has reached the core 206. Thus, the electrode 210 may be used to determine if the system has been sufficiently inserted into the organoid 134. There may be one or more additional sensors 112 disposed on the exterior and/or interior of the cannula 236 to monitor any other suitable parameter of the organoid 134, as described above.

Once the system is inserted in the desired position with the first open end 238 of the cannula 236 disposed in the core 206, nutrients (e.g. liquid media and/or O₂ gas) may be inserted into a second open end 240 of the cannula 236 so that the nutrients can be delivered to the necrotic core 206 via the first open end 238. The second open end 240 of the cannula 236 may be disposed outside of the organoid 134. In some embodiments, waste (e.g. metabolites) produced by the cells in the necrotic core 206 may be removed from the core 206 via the cannula 236.

The cannula 236 may be any suitable size. For example, in some embodiments, the cannula 236 may be approximately 200 micrometers in diameter. Moreover, in some embodiments, the cannula 236 may have a length of approximately 70 millimeters.

In some embodiments, fluid may flow through the cannula 236 (from either the second end 238 to the first end 240 or vice versa) using the concentration gradient between the liquid media and the necrotic core 206, wicking or capillary action, or a pump.

In some embodiments, the cannula 236 may also be used to remove a sample of the necrotic core 206. For example, one or more cells from the necrotic core may be removed via the opening in the cannula 206. In some embodiments, a needle may be inserted through the opening in the cannula 236 or may be inserted adjacent the cannula 236 and a sample may be removed via the needle.

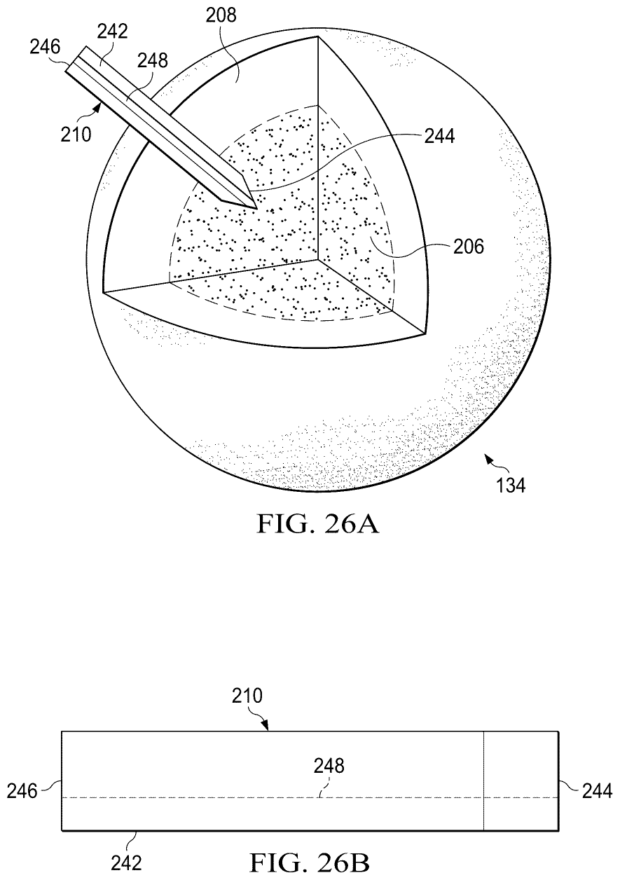

FIGS. 26A-26B illustrate another embodiment of a penetrating electrode 210 for delivering nutrients to the necrotic core 206, according to one or more embodiments of the present disclosure. FIG. 26A illustrates the penetrating electrode 210 inserted into an organoid 134 and FIG. 26B illustrates a side view of the penetrating electrode 210.

In some embodiments, the bottom 242 of the penetrating electrode 210 may be configured to allow fluid flow along it. Thus, when the penetrating electrode 210 is inserted into the necrotic core 206, fluid can be flown from a second end 246 of the penetrating electrode 210 outside of the organoid 134 to a first end 244 of the electrode 210 disposed within the necrotic core 206. In some embodiments, waste produced by cells in the core 206 may be removed from the first end 244 to the second end 246.

This can be accomplished in multiple ways. For example, a divot or channel 248 may be cut into the bottom 242 of the electrode 210. Thus, fluid may be flown through the channel 248. In some embodiments, the bottom 242 may be coated to prevent cell attachment to the bottom 242 and allow for fluid flow thereon.

In some embodiments, the fluid may move along the channel 248 (from either the second end 246 to the first end 244 or vice versa) using the concentration gradient between the liquid media and the necrotic core 206, wicking or capillary action along the bottom 242, or a pump. In some embodiments, an electrode 210 with a channel 248 cut into the bottom 242 may be advantageous because it can provide nutrients to the necrotic core 206 without additional materials or components.

In some embodiments, a porous material may be inserted into the necrotic core 206 of the organoid 134 and fluid may be delivered to the core 206 via the porous material. In some embodiments, waste may also be removed from the core 206 via the porous material.

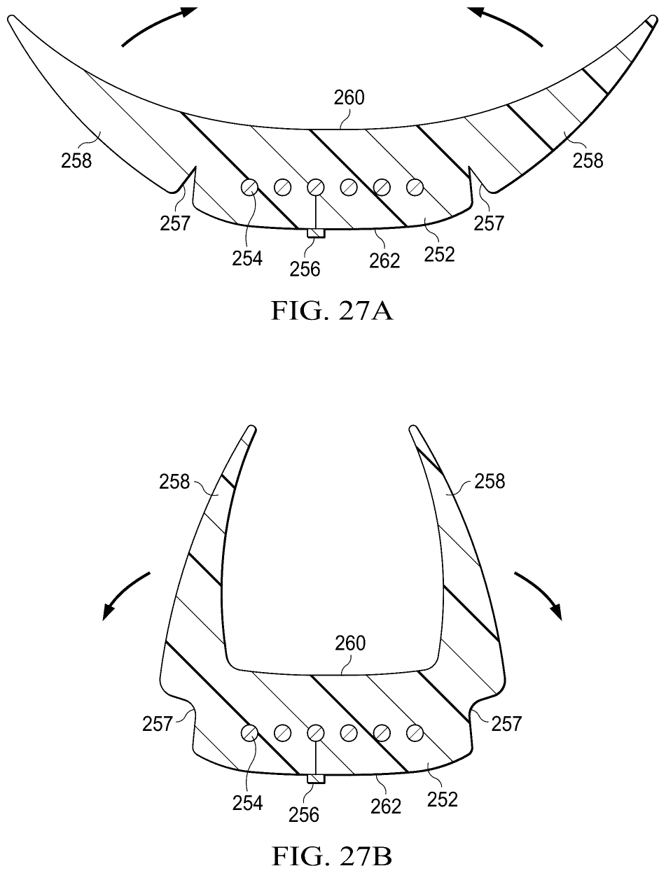

FIGS. 27A-27B illustrate another embodiment for system for delivering fluids and/or removing waste from the necrotic core 206 (not shown in FIGS. 27A-27B) of an organoid 134 (not shown in FIGS. 27A-27B), according to one or more embodiments of the present disclosure. FIGS. 27A and 27B illustrate a cross-section of the system in a first position and a second position, respectively. The system may include a central portion 252 with two side portions 258 on either side of the center portion 252. One or more conductors 254 may extend longitudinally through the center portion 252. Each of the one or more conductors 254 may connect to one or more electrodes 256 disposed on the surface of the central portion 252. Thus, the conductors 254 may be electrically coupled to the electrodes 256.

The side portions 258 may be formed of a conjugated polymer, such as a shape memory polymer, that holds a set shape when a stimulus, such as heat, is applied. In some embodiments, the side portions 258 may be formed of a conjugated polymer that contracts or moves into another position upon application of a voltage. Thus, the side portions 258 may be moveable from a first, uncontracted position to a second, contracted position upon application of a stimulus.

FIG. 27A illustrates the side portions 258 in the uncontracted position. In some embodiments, the side portions 258 and the central portion 252 may be curved (e.g. U-shaped or V-shaped) on a first surface 260 so that a fluid can move along that surface 260. The electrodes 256 may be disposed on an opposite second surface 262.

When a stimulus, such as voltage or heat, is applied to the side portions 258, the side portions contract and move into the contracted position. FIG. 27B illustrates the side portions 258 in the contracted position. In this position, the side portions 258 may move upward and inward towards the first surface 260. There may be a notch or groove 257 between the side portions 258 and the central portion 252 to allow the side portions 260 to more easily bend. The notches 257 may be disposed on the second surface 262 (as shown in FIGS. 27A-27B) and/or on the first surface 260. When the side surfaces 258 contract and/or move into a contracted position, this may push fluid down the length of the central portion 252. This may allow control of the fluid flow along the system into or out of the necrotic core 206.

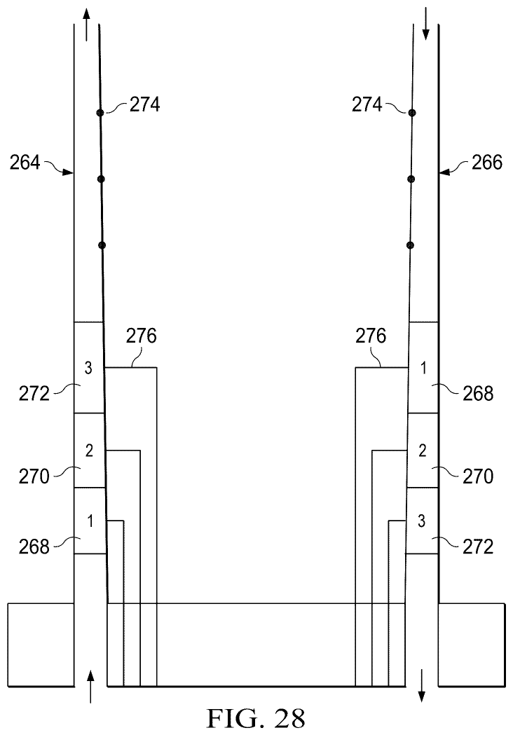

FIG. 28 illustrates another embodiment for system for delivering fluids and/or removing waste from the necrotic core 206 (not shown in FIGS. 27A-27B) of an organoid 134 (not shown in FIGS. 27A-27B), according to one or more embodiments of the present disclosure. The system may include a first cannula 264 and a second cannula 266. The first cannula 264 may be configured to deliver fluids (e.g. nutrients like liquid media) to the necrotic core 206. The second cannula 266 may be configured to remove waste (e.g. metabolites) from the necrotic core 206. Each cannula may include one or more electrodes 274, which may be space longitudinally and/or circumferentially along the cannula 264, 266.

Each cannula 264, 266 may include a first section 268, a second section 270, and third section 272. In some embodiments, the cannulas 264, 266 may include fewer than three sections or may include more than three sections. In some embodiments, the at least part of the cannula 264, 266 may be formed of material that may hold its shape or become rigid upon application of a stimulus. For example, the sections 268, 270, 272 may be formed of a conjugated polymer, such as a shape memory polymer, that changes shape or contracts when a stimulus, such as heat, is applied. Each section 268, 270, 272 may be formed of a conjugated polymer that contracts or shrinks upon application of a voltage. Thus, the sections 268, 270, 272 may change from a first, uncontracted position to a second, contracted position upon application of a stimulus. The stimulus may be applied via one or more electrical leads 276. In some embodiments, each section has its own electrical lead 276.

Each section 268, 270, 272 may be separate such that, when a stimulus is applied to a particular section, the shape changes for that particular section independently of the other sections. For example, when a stimulus is applied to the first section 268, the first section 268 may contract while the other sections 270, 272 remain uncontracted.

Thus, the fluid flow through the cannulas 264, 266 may be controlled by applying a stimulus to the sections sequentially along the length in the direction of fluid flow. In some embodiments, a stimulus may be applied to the first section 268 on the first cannula 264, then to the second section 270 on the first cannula 264, then to the third section 272 on the first cannula 264 so that the first cannula 264 sequentially contracts in a direction towards the core 206. This may cause the fluid to flow in the direction of the arrows shown by the first cannula 264 into the necrotic core 206 to deliver fluids thereto.

Similarly, in some embodiments, a stimulus may be applied to the first section 268 on the second cannula 266, then to the second section 270 on the second cannula 266, then to the third section 272 on the second cannula 266 so that the second cannula 266 sequentially contracts in a direction away from the core 206. This may cause the fluid to flow in the direction of the arrows shown by the second cannula 266 out of the necrotic core 206 to remove fluids therefrom.

In some embodiments, providing nutrients to and removing waste from the necrotic core 206 may reduce the size of the core 206 and increase the number of live/viable cells within the organoid 134. In some embodiments, electrophysiological data and/or biomarker closed-loop feedback measured by the single-unit bioreactor 110 may further be used to minimize the size of the necrotic core 206. Moreover, in some embodiments, one or more of the above described devices may deliver analytes to the necrotic core 206 that may be used for guided localized growth and differentiation factors.

In some embodiments, a multi-functional probe (with a single mode or multiple modes) may be used. The multi-functional probe may be used to control, influence, or guide structural and/or functional organoid growth and maturation. This may affect the similarities and differences within an organoid in an individual well 124 (intra-organoid variability) or between different organoids in the multi-well plate 122 (inter-organoid variability). In some embodiments, an MEA 114 with one or more surface electrodes 212 and/or penetrating electrodes 210 may optimize the multimodal electrophysiological assay. For example, in some embodiments, the MEA 114 may measure impedance or stimulate impedance across gradients.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

Persons of ordinary skill in the art will appreciate that the implementations encompassed by the present disclosure are not limited to the particular exemplary implementations described above. In that regard, although illustrative implementations have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A basket configured to support an organoid, comprising:
a bottom;
a first side extending upward from the bottom; and
a microelectrode array disposed on the bottom.

2. The basket of claim 1, wherein the first side is hemispherical.

3. The basket of claim 1, wherein the first side is cylindrical.

4. The basket of claim 1, further comprising a second side opposite the first side.

5. The basket of claim 1, further comprising one or more perforations in at least said bottom or said first side.

6. The basket of claim 1, wherein the microelectrode array comprises one or more electrodes configured to penetrate the organoid.

7. The basket of claim 1, wherein the microelectrode array comprises one or more electrodes configured to contact a surface of the array.

8. A bioreactor system for organoids, comprising:
one or more single-unit bioreactors, comprising:
a basket shaped to hold a three-dimensional organoid;
a microelectrode array disposed within the basket; and
one or more sensors;
a fluid handling system, comprising:
a first holding chamber for a first fluid;
a dispensing tip coupled to the first holding chamber and configured to deliver the first fluid to at least one of the one or more single-unit bioreactors; and
a processor in communication with the microelectrode array and the one or more sensors, wherein the processor is configured to receive a first plurality of electrical signals from the one or more sensors.

9. The bioreactor system of claim 8, wherein the processor is further configured to receive a second plurality of electrical signals from the microelectrode array.

10. The bioreactor system of claim 8, wherein the processor is further configured to send a third plurality of electrical signals to the microelectrode array.

11. The bioreactor system of claim 8, wherein the processor is further in communication with the fluid handling system.

12. The bioreactor system of claim 11, wherein the processor is further configured to control the fluid handling system to dispense a particular amount of the first fluid into a first single-unit bioreactor of the one or more single-unit bioreactors.

13. The bioreactor system of claim 8, wherein the fluid handling system further comprises a second holding chamber for holding a second fluid, wherein the dispensing tip is further configured to deliver the second fluid to at least one of the one or more single-unit bioreactors.
